(19) United States
(12) Patent Application Publication
(10) Pub. No.: US 2026/0325707 A1
(43) Pub. Date: Oct. 22, 2026

(54) DISSOLVABLE iEDDA CLICK HYDROGEL AND METHODS OF MAKING AND USING THE SAME

(71) Applicant: The Trustees of Indiana University, Bloomington, IN (US)

(72) Inventor: Chien-Chi Lin, Indianapolis, IN (US)

(21) Appl. No.: 19/116,497

(22) PCT Filed: Apr. 25, 2023
(86) PCT No.: PCT/US2023/066190

§ 371 Date: Mar. 28, 2025

Related U.S. Application Data
(60) Provisional application No. 63/411,385, filed on Sep. 29, 2022.

(51) Int. Cl. A61L 26/00; A61K 9/50

(52) U.S. Cl. CPC A61L 26/008; A61K 9/5089; A61L 26/0019; A61L 26/0066

(57) ABSTRACT

A composition comprising a hydrogel includes a polymer of formula (1), at least one repeating unit of which comprises norborene. Methods of treating wounds and methods of encapsulating drugs and/or cells using such hydrogels are also disclosed herein.

### CROSS-REFERENCE TO RELATED APPLICATION

This application claims priority to U.S. provisional patent application No. 63/411,385, filed Sep. 29, 2022, the entire content of which is incorporated by reference herein.

### STATEMENT REGARDING FEDERALLY SPONSORED RESEARCH

This invention was made with government support under CA227737 awarded by National Institutes of Health. The Government has certain rights in the invention.

### FIELD

The present disclosure provides hydrogels and methods of making and using the same.

### BACKGROUND

Hydrogels have shown promise as matrices for three-dimensional cell culture. Cells tend to adhere, spread, migrate and proliferate better in hydrogels with pronounced stress relaxation. Hydrogels have other potential uses depending on the tunability of certain properties of the hydrogels, including in wound treatment, drug encapsulation, and cellular encapsulation.

### SUMMARY

A need exists, therefore, for mechanisms for tuning the stress relaxation and other viscoelastic behaviors of hydrogels.

The present disclosure provides a composition comprising a hydrogel precursor, the hydrogel precursor comprising: a first reaction product of a hydroxyl-terminated first multi-arm polyethylene glycol and a compound selected from the group consisting of a carbic anhydride, a norbornene acid, and a combination thereof; and a second reaction product of a tetrazine amine and a second multi-arm polyethylene glycol comprising an amido succinic acid moiety.

In some embodiments, the present disclosure provides a wound dressing comprising: a hydrogel formed from a hydrogel precursor, the hydrogel precursor comprising: a first reaction product of a hydroxyl-terminated first multi-arm polyethylene glycol and a compound selected from the group consisting of a carbic anhydride, a norbornene acid, and a combination thereof; and a second reaction product of a tetrazine amine and a second multi-arm polyethylene glycol comprising an amido succinic acid moiety.

In some embodiments, the present disclosure provides a method for manufacturing a wound dressing comprising: forming a composition comprising a hydrogel precursor, the hydrogel precursor comprising a first reaction product of a hydroxyl-terminated first multi-arm polyethylene glycol and a compound selected from the group consisting of a carbic anhydride, a norbornene acid, and a combination thereof; and a second reaction product of a tetrazine amine and a second multi-arm polyethylene glycol comprising an amido succinic acid moiety; allowing the tetrazine amine of the second reaction product to react with the first reaction product to form the hydrogel, thereby forming a hydrogel; and applying the hydrogel to a support, thereby forming the wound dressing.

In some embodiments, the present disclosure provides a method for using a wound dressing comprising a hydrogel formed from a hydrogel precursor, the hydrogel precursor comprising: a first reaction product of a hydroxyl-terminated first multi-arm polyethylene glycol and a compound selected from the group consisting of a carbic anhydride, a norbornene acid, and a combination thereof; and a second reaction product of a tetrazine amine and a second multi-arm polyethylene glycol comprising an amido succinic acid moiety. The method includes allowing the tetrazine amine of the second reaction product to react with the first reaction product to form the hydrogel; applying the hydrogel to a support, thereby forming the wound dressing; and applying the wound dressing to a wound.

In some embodiments, the present disclosure provides method for treating a wound in a subject in need thereof, the method comprising: forming the composition disclosed above; applying the composition to the wound; and allowing the tetrazine amine of the second reaction product to react with the first reaction product to form the hydrogel, thereby treating the wound.

In some embodiments, the present disclosure provides method for encapsulating a drug, a cell, or both a drug and a cell, the method comprising: forming the composition disclosed above; mixing the drug, the cell, or both the drug and the cell, with the composition; and allowing the tetrazine amine of the second reaction product to react with the first reaction product to form the hydrogel, thereby encapsulating the drug, the cell, or both the drug and the cell.

This summary is not intended to identify key or essential features of the claimed subject matter, nor is it intended to be used as an aid in limiting the scope of the claimed subject matter. Further embodiments, forms, features, and aspects of the present disclosure shall become apparent from the description and figures provided herewith.

### BRIEF DESCRIPTION OF THE DRAWINGS

The teachings of some embodiments of the present disclosure will be better understood by reference to the description taken in conjunction with the accompanying drawings, wherein:

FIG. 1 shows ¹H NMR spectra of (a) polyethylene glycol-norbornene-carbic anhydride (PEGNB_{CA}) and (b) polyethylene glycol-norbornene (PEGNB);

FIG. 2 shows in situ rheometry of inverse electron demand Diels-Alder click chemistry (iEDDA) click hydrogel crosslinking using PEGNB_{CA} with PEG-tetrazine (PEGTz) or PEG-methyltetrazine (PEGmTz);

FIG. 3 shows shear moduli of 2.5 wt % PEGNB_{CA} or PEGNB crosslinked with either PEGTz or PEGmTz (R=1). ***p<0.001;

FIG. 4 shows characterization of hydrogel degradation via shear moduli change (i.e., G′/G′₀) as a function of time (curve fitting represents best-fit of pseudo-first order hydrolytic degradation kinetics);

FIG. 5 shows gel fraction of 2.5 wt % PEGNB_{CA} crosslinked with PEGTz and PEGmTz over 28 day period;

FIG. 6 shows swelling ratio of 2.5 wt % PEGNB_{CA} crosslinked with PEGTz and PEGmTz over 28 day period;

FIG. 7 shows ¹H NMR spectra of PEGmTz/Tz ratios;

FIG. 8 shows schematics of methyltetrazine/tetrazine (mTz/Tz) dual-funcitonalized PEG macromers;

FIG. 9 shows in situ rheometry of PEGNB or PEGNB_{CA} (2.5 wt %) gel crosslinking using PEG-50 mTz/50 Tz at 37° C. (R=1);

FIG. 10 shows in situ rheometry of PEGNB_{CA} (2.5 wt %) gel crosslinking using PEG-mTz/Tz at different mTz/Tz ratios at 37° C.;

FIG. 11 shows in situ rheometry of PEGNB_{CA} (2.5 wt %) gel crosslinking using PEG-50 mTz/50 Tz at 25° C. or 37° C. (R=1);

FIG. 12 shows effect of PEGNB_{CA} wt % on shear moduli of iEDDA hydrogels with different PEGmTz/Tz crosslinks (R=1);

FIG. 13 shows effect of (m)Tz/NB molar ratio on shear moduli of PEGNB_{CA} hydrogels with different PEGmTz/Tz macromers (hydrogel shear moduli were measured 1 hour after swelling);

FIG. 14 shows gel fraction of 2.5 wt % PEGNB_{CA} with PEGmTz/Tz ratios;

FIG. 15 shows swelling ratio of 2.5 wt % PEGNB_{CA} with PEGmTz/Tz ratios;

FIG. 16 shows calculated mesh size of 2.5 wt % PEGNB_{CA} with PEGmTz/Tz ratios;

FIG. 17 shows change in initial shear moduli of PEGNB_{CA} over 4 days with different PEGmTzTz crosslinkers based on different formulations: (a) 1.75 wt %, (b) 2.5 wt %, and (c) 4 wt % of PEGNB_{CA} fixed at R=1, and (d) R=2, and (e) R=0.5 fixed at 2.5 wt % PEGNB_{CA};

FIG. 18 shows a schematic of hydrolytic degradation of PEGNB_{CA}-based iEDDA click hydrogels;

FIG. 19 shows hydrolytic degradation of PEGNB_{CA}-based iEDDA click hydrogels (PEGNB_{CA} was crosslinked with PEG75mTz/25Tz, ratios at 2.5 wt % with R at 0.5, 1.0, and 2.0; values of G′₀ were obtained on day 0 after 1 hour of swelling);

FIG. 20 shows hydrolytic degradation of PEGNB_{CA}-based iEDDA click hydrogels (PEGNB_{CA} was crosslinked with PEG50 mTz/50 Tz, ratios at 2.5 wt % with R at 0.5, 1.0, and 2.0; values of G′₀ were obtained on day 0 after 1 hour of swelling);

FIG. 21 shows hydrolytic degradation of PEGNB_{CA}-based iEDDA click hydrogels (PEGNB_{CA} was crosslinked with PEG25mTz75Tz, ratios at 2.5 wt % with R at 0.5, 1.0, and 2.0; values of G′₀ were obtained on day 0 after 1 hour of swelling);

FIG. 22 shows hydrolytic degradation of PEGNB_{CA}-based iEDDA click hydrogels (PEGNB_{CA} was crosslinked with PEG75mTz/25Tz at different weight percents (1.75 wt %, 2.5 wt %, and 4 wt %) at R=1 (values of G′₀ were obtained on day 0 after 1 hour of swelling);

FIG. 23 shows hydrolytic degradation of PEGNB_{CA}-based iEDDA click hydrogels (PEGNB_{CA} was crosslinked with PEG50 mTz/50 Tz at different weight percents (1.75 wt %, 2.5 wt %, and 4 wt %) at R=1 (values of G′₀ were obtained on day 0 after 1 hour of swelling);

FIG. 24 shows hydrolytic degradation of PEGNB_{CA}-based iEDDA click hydrogels (PEGNB_{CA} was crosslinked with PEG-25mTz/75Tz at different weight percents (1.75 wt %, 2.5 wt %, and 4 wt %) at R=1 (values of G′₀ were obtained on day 0 after 1 hour of swelling);

FIG. 25 shows swelling ratio of 2.5 wt % PEGNB_{CA} with PEGmTz/Tz ratios over time;

FIG. 26 shows representative confocal images of live/dead stained human mesenchymal stem cells (hMSCs) encapsulated in 2.5 wt % PEGNB_{CA} based iEDDA hydrogels crosslinked with PEG-25mTz/75Tz at R=0.5 with 3 wt % Gel-50 mTz/50 Tz;

FIG. 27 shows representative F-actin staining confocal images (day 14) in which hMSCs were encapsulated in 2.5 wt % PEGNB_{CA};

FIG. 28 shows circularity results obtained from the F-actin staining of FIG. 27;

FIG. 29 shows aspect ratio results obtained from the F-actin staining of FIG. 27;

FIG. 30 shows proteolytic degradation measured by hydrogel mass over 3 hours of 2.5 wt % PEGNB or PEGNB_{CA} crosslinked with PEG25mTz/75Tz at R=0.5 with 3 wt % Gel-50 mTz/50 Tz in the presence of type 1 collagenase at 30 U/mL;

FIG. 31 shows shear moduli overtime of 2.5 wt % PEGNB or PEGNB_{CA} at R=0.5 with 3 wt % Gel-50 mTz/50 Tz;

FIG. 32 shows microscope images of immunohistology samples containing PEGNB hydrogel: (A) hematoxylin and eosin Y (H&E) and (B) CD68;

FIG. 33 shows in vivo tissue response to injectable PEG-based iEDDA hydrogels (14-day post-injection, tissues were explanted and subjected to immunohistochemical staining using hematoxylin and eosin Y (H&E). Hydrogels were crosslinked by 2.5 wt % PEGNB or PEGNB_{CA} with PEG-25mTz/75Tz at R=0.8 with 3 wt % Gel-50 mTz/50 Tz);

FIG. 34 shows in vivo tissue response to injectable PEG-based iEDDA hydrogels (14-day post-injection, tissues were explanted and subjected to immunohistochemical staining using anti-CD68 and anti-CD45 antibodies. Hydrogels were crosslinked by 2.5 wt % PEGNB or PEGNB_{CA} with PEG-25mTz/75Tz at R=0.8 with 3 wt % Gel-50 mTz/50 Tz);

FIG. 35 shows image analysis of microscope images of mouse tissue samples at injection site stained with CD68 and CD45; and

FIG. 36 shows shear moduli overtime of 2.5 wt % PEGNB or PEGNB_{CA} at R=0.8 with 3 wt % Gel-50 mTz/50 Tz.

### DETAILED DESCRIPTION

The embodiments described below are not intended to be exhaustive or to limit the invention to the precise forms disclosed in the following detailed description. Rather, the embodiments are chosen and described so that others skilled in the art may appreciate and understand the principles and practices of this disclosure.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination.

Unless defined otherwise, all technical and scientific terms have the same meaning as is commonly understood by one of ordinary skill in the art to which this disclosure belongs. All patents, applications, published applications and other publications are incorporated by reference in their entireties. If a definition set forth in this section is contrary to, or otherwise inconsistent with, a definition set forth in a patent, application, or other publication that is incorporated by reference, the definition set forth in this section prevails over the definition incorporated by reference.

As used in the description and the appended claims, the singular forms “a,” “an,” and “the” are intended to include the plural forms as well, unless the context clearly indicates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. The terms “including,” “containing,” and “comprising” are used in their open, non-limiting sense. Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount of polypeptide, dose, time, temperature, enzymatic activity or other biological activity and the like, is meant to encompass variations of ±20%, ±10%, ±5%, ±1%, ±0.5%, or even ±0.1% of the specified amount. To provide a more concise description, some of the quantitative expressions are not qualified with the term “about.” It is understood that, whether the term “about” is used explicitly or not, every quantity is meant to refer to the actual given value, and it is also meant to refer to the approximation to such given value that would reasonably be inferred based on the ordinary skill in the art, including equivalents and approximations due to the experimental and/or measurement conditions for such given value.

The terms “including,” “containing,” and “comprising” are used in their open, non-limiting sense. The transitional phrase “consisting essentially of” means that the scope of a claim is to be interpreted to encompass the specified materials or steps recited in the claim, “and those that do not materially affect the basic and novel characteristic(s)” of the claimed subject matter. See In re Herz, 537 F.2d 549, 551-52, 190 USPQ 461, 463 (CCPA 1976) (emphasis in the original); see also MPEP § 2111.03 (9^{th} edition, 10^{th} revision).

Certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable subcombination. All combinations of the embodiments pertaining to the compositions represented by the variables are specifically embraced by the present disclosure just as if each and every combination was individually and explicitly disclosed. In addition, all subcombinations of the chemical groups listed in the embodiments describing such variables are also specifically embraced by the present disclosure just as if each and every such sub-combination of composition was individually and explicitly disclosed.

References in the specification to “one embodiment,” “an embodiment,” “an illustrative embodiment,” etc., indicate that the embodiment described may include a particular feature, structure, or characteristic, but every embodiment may or may not necessarily include that particular feature, structure, or characteristic. Moreover, such phrases are not necessarily referring to the same embodiment. It should be further appreciated that although reference to a “preferred” component or feature may indicate the desirability of a particular component or feature with respect to an embodiment, the disclosure is not so limiting with respect to other embodiments, which may omit such a component or feature. Further, when a particular feature, structure, or characteristic is described in connection with an embodiment, it is submitted that it is within the knowledge of one skilled in the art to implement such feature, structure, or characteristic in connection with other embodiments whether or not explicitly described. Additionally, it should be appreciated that items included in a list in the form of “at least one of A, B, and C” can mean (A); (B); (C); (A and B); (B and C); (A and C); or (A, B, and C). Similarly, items listed in the form of “at least one of A, B, or C” can mean (A); (B); (C); (A and B); (B and C); (A and C); or (A, B, and C). Further, with respect to the claims, the use of words and phrases such as “a,” “an,” “at least one,” and/or “at least one portion” should not be interpreted so as to be limiting to only one such element unless specifically stated to the contrary, and the use of phrases such as “at least a portion” and/or “a portion” should be interpreted as encompassing both embodiments including only a portion of such element and embodiments including the entirety of such element unless specifically stated to the contrary.

As used herein, the term “mammal” refers to, for example, humans, other primates (e.g., monkeys, chimpanzees, etc.), companion animals (e.g., dogs, cats, horses, etc.), farm animals (e.g., goats, sheep, pigs, cattle, etc.), laboratory animals (e.g., mice, rats, etc.), and wild and zoo animals (e.g., wolves, bears, deer, etc.).

As used herein, the term “drug” refers to a therapeutically active compound, as well as any prodrugs thereof and pharmaceutically acceptable salts, hydrates, and solvates of the compound and the prodrugs. Additional active ingredients may be combined with a compound of the present disclosure and may be either administered separately or in the same pharmaceutical composition. The amount of additional active ingredients to be given may be determined by one skilled in the art based upon therapy with a compound of the present disclosure. As used herein, the term “pharmaceutically acceptable,” with respect to salts and formulation components such as carriers, excipients, and diluents, refers to those salts and components which are not deleterious to a patient and which are compatible with other ingredients, active ingredients, salts or components. Pharmaceutically acceptable includes “veterinarily acceptable,” and thus includes both human and non-human mammal applications independently. As used herein, the term “pharmaceutically acceptable salt” refers to salts commonly used to form alkali metal salts and to form addition salts of free acids or free bases. Such salts include, for example, the physiologically acceptable salts listed in Handbook of Pharmaceutical Salts: Properties, Selection and Use, P. H. Stahl and C. G. Wermuth (Eds.), Wiley-VCH, New York, 2002, which are known to the skilled artisan. Salt formation can occur at one or more positions having labile protons. The pharmaceutically acceptable salts of a compound of the present disclosure include both acid addition salts and base addition salts.

Nuclear magnetic resonance (NMR) spectroscopy is a technique for determining the molecular structure and concentration of molecules. In the NMR analysis of compositions described herein, the compositions or one or more components thereof are maintained in solution and then placed in a large magnetic field. The magnetic field is typically generated by a cryogenically cooled superconducting magnet, but lower magnetic fields including those generated by room temperature electromagnets or rare earth magnets can also be used. The samples are exposed to a band of radiofrequency (RF) waves which are absorbed by the molecules and excite the nuclei of the molecules. The typical focus of these experiments is on the excitation of hydrogen nuclei (also referred to as protons) and nuclei of carbon, in particular the C-13 isotope. After the RF excitation is turned off, a receiver coil then detects the RF energy that is released by the nuclei as they return from the excited state. Following Fourier transformation of these signals, an NMR spectrum is generated containing spectral peaks that, in different combinations, represent the identity of the molecules in solution. The resulting NMR spectrum can then be compared with reference spectra to both identify and quantify the molecules in the solution.

Gel permeation chromatography (GPC), also sometimes referred to interchangeably as gas permeation chromatography, is an analytical method to characterize molecular weights (MW) and dispersity index (Ð) of polymers. GPC is based on size-exclusion chromatography, where the polymers are loaded on the column and separated based on their hydrodynamic volume. Principally, porous beads are packed in the GPC column and the polymers are eluted by organic solvent through the column. Smaller polymers can enter the pores of the beads more easily than larger polymers, hence increasing their retention time in the column. As such, larger polymers are eluted out first while smaller polymers are retained in the column for a longer time. Based on the retention time and elution profiles, polymer molecular weights and dispersity can be calculated.

In an aspect of the disclosure, there is provided a composition comprising a hydrogel precursor, the hydrogel precursor comprising a first reaction product of a hydroxyl-terminated first multi-arm polyethylene glycol and a compound selected from the group consisting of a carbic anhydride, a norbornene acid, and a combination thereof; and a second reaction product of a tetrazine amine and a second multi-arm polyethylene glycol comprising an amido succinic acid moiety. An exemplary embodiment of the composition from which a hydrogel has been formed is shown in Scheme 1. In Scheme 1, R may be H (“unsubstituted tetrazine”) or R may be —CH₃ (“methyltetrazine”).

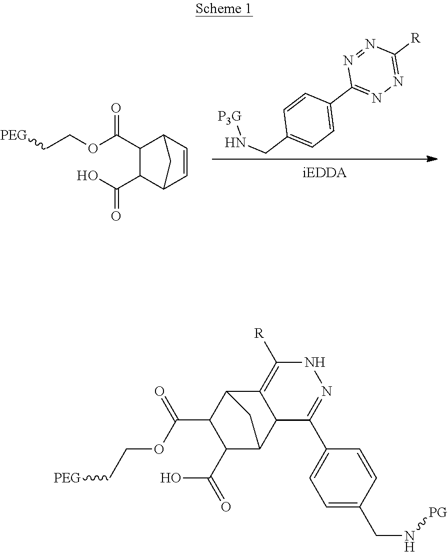

In embodiments, the composition may comprise a first reaction product of a hydroxyl-terminated first multi-arm polyethylene glycol and a compound selected from the group consisting of a carbic anhydride, a norbornene acid, and a combination thereof, as shown in Scheme 2.

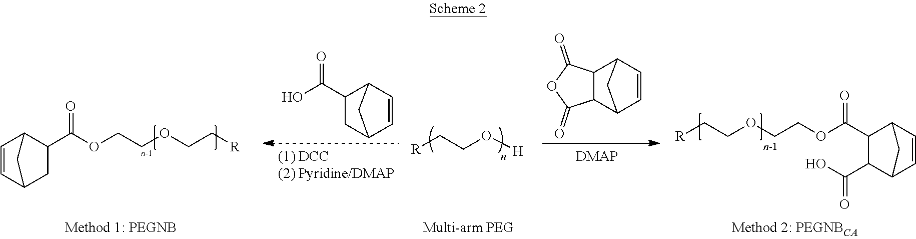

As noted above, the composition further includes a second reaction product of a tetrazine amine and a second multi-arm polyethylene glycol comprising an amido succinic acid moiety, as shown in Scheme 3.

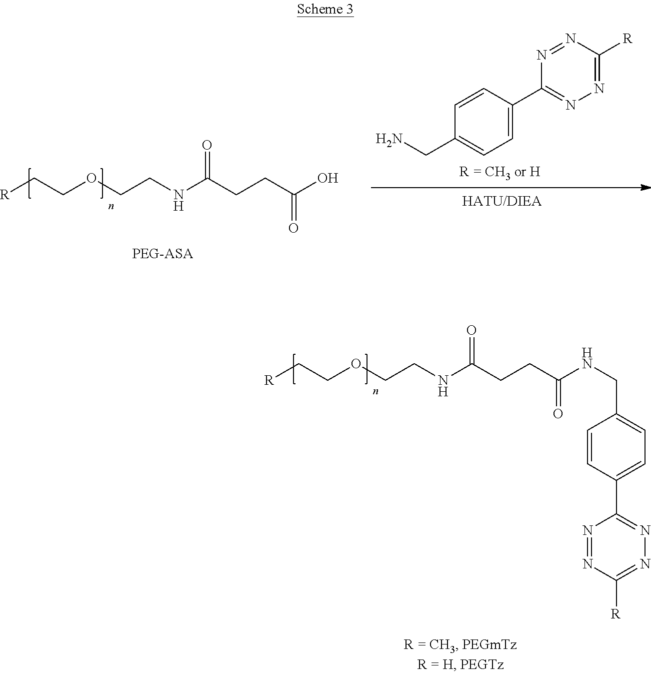

In embodiments, the first multi-arm polyethylene glycol may comprise greater than one arm, such as two arms, three arms, four arms, five arms, six arms, seven arms, or eight arms. In embodiments, the second multi-arm polyethylene glycol may comprise greater than one arm, such as two arms, three arms, four arms, five arms, six arms, seven arms, or eight arms. In embodiments, the second multi-arm polyethylene glycol may comprise four arms, all four of which comprise an unsubstituted tetrazine amine. In embodiments, the second multi-arm polyethylene glycol may comprise four arms, all four of which comprise a methyltetrazine amine. In embodiments, the second multi-arm polyethylene glycol may comprise four arms, three of which comprise an unsubstituted tetrazine amine and the fourth of which comprises methyltetrazine amine. In embodiments, the second multi-arm polyethylene glycol may comprise four arms, two of which comprise an unsubstituted tetrazine amine and the other two of which comprise methyltetrazine amine. In embodiments, the second multi-arm polyethylene glycol may comprise four arms, one of which comprises an unsubstituted tetrazine amine and the other three of which comprise methyltetrazine amine.

In embodiments, each arm of the first multi-arm polyethylene glycol and the second multi-arm polyethylene glycol may have a number of repeating units, n, that is an integer from 1 to 5000. For example, n may each independently be an integer from 1 to 4500, 1 to 4000, 2 to 4000, 1 to 3500, from 1 to 3000, from 1 to 2500, from 1 to 2000, from 1 to 1500, from 1 to 1000, from 1 to 500, from 1 to 450, from 1 to 400, from 1 to 350, from 1 to 300, from 1 to 250, from 1 to 200, from 1 to 150, from 1 to 100, from 1 to 50, from 50 to 5000, from 100 to 5000, from 150 to 5000, from 200 to 5000, from 250 to 5000, from 300 to 5000, from 350 to 5000, from 400 to 5000, from 450 to 5000, from 500 to 5000, from 1000 to 5000, from 1500 to 5000, from 2000 to 5000, from 2500 to 5000, from 3000 to 5000, from 3500 to 5000, or even from 4000 to 5000.

In embodiments, the first multi-arm polyethylene glycol and the second multi-arm polyethylene glycol may have a number average molecular weight (MN), obtained via GPC, from 70 kDa to 250 kDa. That is, the MN may be from 75 kDa to 250 kDa, from 80 kDa to 250 kDa, from 85 kDa to 250 kDa, from 90 kDa to 250 kDa, from 95 kDa to 250 kDa, from 100 kDa to 250 kDa, from 105 kDa to 250 kDa, from 110 kDa to 250 kDa, from 115 kDa to 250 kDa, from 120 kDa to 250 kDa, from 125 kDa to 250 kDa, from 130 kDa to 250 kDa, from 135 kDa to 250 kDa, from 140 kDa to 250 kDa, from 145 kDa to 250 kDa, from 150 kDa to 250 kDa, from 155 kDa to 250 kDa, from 160 kDa to 250 kDa, from 165 kDa to 250 kDa, from 170 kDa to 250 kDa, from 175 kDa to 250 kDa, from 180 kDa to 250 kDa, from 185 kDa to 250 kDa, from 190 kDa to 250 kDa, from 195 kDa to 250 kDa, from 200 kDa to 250 kDa, from 205 kDa to 250 kDa, from 210 kDa to 250 kDa, from 215 kDa to 250 kDa, from 220 kDa to 250 kDa, from 225 kDa to 250 kDa, from 230 kDa to 250 kDa, from 235 kDa to 250 kDa, from 240 kDa to 250 kDa, from 245 kDa to 250 kDa, from 70 kDa to 245 kDa, from 70 kDa to 240 kDa, from 70 kDa to 235 kDa, from 70 kDa to 230 kDa, from 70 kDa to 225 kDa, from 70 kDa to 220 kDa, from 70 kDa to 215 kDa, from 70 kDa to 210 kDa, from 70 kDa to 205 kDa, from 70 kDa to 200 kDa, from 70 kDa to 195 kDa, from 70 kDa to 190 kDa, from 70 kDa to 185 kDa, from 70 kDa to 180 kDa, from 70 kDa to 175 kDa, from 70 kDa to 170 kDa, from 70 kDa to 165 kDa, from 70 kDa to 160 kDa, from 70 kDa to 155 kDa, from 70 kDa to 150 kDa, from 70 kDa to 145 kDa, from 70 kDa to 140 kDa, from 70 kDa to 135 kDa, from 70 kDa to 130 kDa, from 70 kDa to 125 kDa, from 70 kDa to 120 kDa, from 70 kDa to 115 kDa, from 70 kDa to 110 kDa, from 70 kDa to 105 kDa, from 70 kDa to 100 kDa, from 70 kDa to 95 kDa, from 70 kDa to 90 kDa, from 70 kDa to 85 kDa, from 70 kDa to 80 kDa, or even from 70 kDa to 75 kDa. Without intending to be bound by any particular theory, it is believed that hydrogel crosslinking may be inefficient if the MN is too low. Further, it is believed that too high of a MN may limit the useability of hydrogels in vivo.

The reaction product of the hydroxyl-terminated first multi-arm polyethylene glycol and the carbic anhydride or the reaction product of the hydroxyl-terminated first multi-arm polyethylene glycol and the norbornene acid can undergo an inverse electron demand Diels-Alder reaction (iEDDA) with the reaction product of the second multi-arm polyethylene glycol comprising the amido succinic acid and a tetrazine amine, as shown in Scheme 1, above. In embodiments, the thus produced hydrogel may be used to manufacture a wound dressing. For example, a wound dressing may include a hydrogel formed from a hydrogel precursor, the hydrogel precursor comprising: a first reaction product of a hydroxyl-terminated first multi-arm polyethylene glycol and a compound selected from the group consisting of a carbic anhydride, a norbornene acid, and a combination thereof; and a second reaction product of a tetrazine amine and a second multi-arm polyethylene glycol comprising an amido succinic acid moiety.

In embodiments, the composition comprising the hydrogel may have a shear modulus from 1 kPa to 100 kPa. That is, the shear modulus may be from 1 kPa to 90 kPa, from 1 kPa to 85 kPa, from 1 kPa to 80 kPa, from 1 kPa to 75 kPa, from 1 kPa to 70 kPa, from 1 kPa to 65 kPa, from 1 kPa to 60 kPa, from 1 kPa to 55 kPa, from 1 kPa to 50 kPa, from 1 kPa to 45 kPa, from 1 kPa to 40 kPa, from 1 kPa to 35 kPa, from 1 kPa to 30 kPa, from 1 kPa to 25 kPa, from 1 kPa to 20 kPa, from 1 kPa to 15 kPa, from 1 kPa to 10 kPa, from 1 kPa to 5 kPa, from 10 kPa to 100 kPa, from 15 to 100 kPa, from 20 kPa to 100 kPa, from 25 to 100 kPa, from 30 kPa to 100 kPa, from 35 to 100 kPa, from 40 kPa to 100 kPa, from 45 to 100 kPa, from 50 kPa to 100 kPa, from 55 to 100 kPa, from 60 kPa to 100 kPa, from 65 to 100 kPa, from 70 kPa to 100 kPa, from 75 to 100 kPa, from 80 kPa to 100 kPa, from 85 to 100 kPa, from 90 to 100 kPa, or even from 95 kPa to 100 kPa. Without intending to be bound by any particular theory, it is believed that an undesirably weak hydrogel may be obtained when the shear modulus is too low. Further, it is believed that the hydrogel may be too stiff if the shear modulus is too high. Hydrogels that are either too weak or too stiff are not physiologically relevant.

In embodiments, the composition comprising the hydrogel may have an elastic modulus (G′) from 1 kPa to 100 kPa. That is, G′ may be from 1 kPa to 90 kPa, from 1 kPa to 85 kPa, from 1 kPa to 80 kPa, from 1 kPa to 75 kPa, from 1 kPa to 70 kPa, from 1 kPa to 65 kPa, from 1 kPa to 60 kPa, from 1 kPa to 55 kPa, from 1 kPa to 50 kPa, from 1 kPa to 45 kPa, from 1 kPa to 40 kPa, from 1 kPa to 35 kPa, from 1 kPa to 30 kPa, from 1 kPa to 25 kPa, from 1 kPa to 20 kPa, from 1 kPa to 15 kPa, from 1 kPa to 10 kPa, from 1 kPa to 5 kPa, from 10 kPa to 100 kPa, from 15 to 100 kPa, from 20 kPa to 100 kPa, from 25 to 100 kPa, from 30 kPa to 100 kPa, from 35 to 100 kPa, from 40 kPa to 100 kPa, from 45 to 100 kPa, from 50 kPa to 100 kPa, from 55 to 100 kPa, from 60 kPa to 100 kPa, from 65 to 100 kPa, from 70 kPa to 100 kPa, from 75 to 100 kPa, from 80 kPa to 100 kPa, from 85 to 100 kPa, from 90 to 100 kPa, or even from 95 kPa to 100 kPa. Without intending to be bound by any particular theory, it is believed that an undesirably weak hydrogel may be obtained when G′ is too low. Further, it is believed that the hydrogel may be too stiff if G′ is too high. Hydrogels that are either too weak or too stiff are not physiologically relevant.

In embodiments, the hydrogel may have a tan δ from 0.01 to 0.15. That is, the tan δ may be from 0.01 to 0.14, from 0.01 to 0.13, from 0.01 to 0.12, from 0.01 to 0.11, from 0.01 to 0.1, from 0.01 to 0.09, from 0.01 to 0.08, from 0.01 to 0.07, from 0.01 to 0.06, from 0.01 to 0.05, from 0.01 to 0.04, from 0.01 to 0.03, from 0.01 to 0.02, from 0.02 to 0.15, from 0.03 to 0.15, from 0.04 to 0.15, from 0.05 to 0.15, from 0.06 to 0.15, from 0.07 to 0.1, from 0.08 to 0.15, from 0.09 to 0.15, from 0.1 to 0.15, from 0.11 to 0.15, from 0.12 to 0.15, from 0.13 to 0.15, or even from 0.14 to 0.15. Without intending to be bound by any particular theory, tan δ is a measurement of hydrogel viscoelasticity. Similar to G′, there is an optimal range for soft tissues. Materials with too low or too high tan δ do not mimic tissues in the body, and are therefore not physiologically relevant.

In embodiments, the hydrogel may have a stress-relaxation halftime (t_{1/2}) from 10 seconds to 100 seconds. That is, the t_{1/2} may be from 10 seconds to 90 seconds, from 10 seconds to 85 seconds, from 10 seconds to 80 seconds, from 10 seconds to 75 seconds, from 10 seconds to 70 seconds, from 10 seconds to 65 seconds, from 10 seconds to 60 seconds, from 10 seconds to 55 seconds, from 10 seconds to 50 seconds, from 10 seconds to 45 seconds, from 10 seconds to 40 seconds, from 10 seconds to 35 seconds, from 10 seconds to 30 seconds, from 10 seconds to 25 seconds, from 10 seconds to 20 seconds, from 10 seconds to 15 seconds, from 20 seconds to 100 seconds, from 25 seconds to 100 seconds, from 30 seconds to 100 seconds, from 35 seconds to 100 seconds, from 40 seconds to 100 seconds, from 45 seconds to 100 seconds, from 50 seconds to 100 seconds, from 55 seconds to 100 seconds, from 60 seconds to 100 seconds, from 65 seconds to 100 seconds, from 70 seconds to 100 seconds, from 75 seconds to 100 seconds, from 80 seconds to 100 seconds, from 85 seconds to 100 seconds, from 90 seconds to 100 seconds, or even from 95 seconds to 100 seconds. Without intending to be bound by any particular theory, t_{1/2} is a measurement of hydrogel viscoelasticity. Similar to G′ and tan δ, there is an optimal range for soft tissues. Materials with too low or too high t_{1/2} do not mimic tissues in the body, and are therefore not physiologically relevant.

In embodiments, the hydrogel may have a hydrolytic half-life from 5 days to 75 days. That is the composition may have a hydrolytic half-life from 5 days to 70 days, from 5 days to 65 days, from 5 days to 60 days, from 5 days to 55 days, from 5 days to 50 days, from 5 days to 45 days, from 5 days to 40 days, from 5 days to 35 days, from 5 days to 30 days, from 5 days to 25 days, from 5 days to 20 days, from 5 days to 15 days, from 5 days to 10 days, from 5 days to 70 days, from 10 days to 75 days, from 15 days to 75 days, from 20 days to 75 days, from 25 days to 75 days, from 30 days to 75 days, from 35 days to 75 days, from 40 days to 75 days, from 45 days to 75 days, from 50 days to 75 days, from 50 days to 70 days, from 55 days to 75 days, from 60 days to 75 days, from 65 days to 75 days, or even from 70 days to 75 days. Without intending to bound by any particular theory, if the hydrolytic half-life is too short or too long, the resulting gels are less than optimal for their intended purposes.

In embodiments, the composition may include a tissue adhesive moiety, which adheres to the surfaces of the wound being treated to aid in the healing process. In embodiments, the adhesive may be prepared from dissolvable hydrogels crosslinked by an inverse electron-demand Diels-Alder (iEDDA) reaction between norbornene-Dopa-derivatized PEG (PEGNB-D) and tetrazine-derivatized PEG (PEGTz). Without intending to be bound by any particular theory, it is believed that the tissue adhesiveness is provided by the Dopa moiety conjugated on a PEG macromer modified with carbic anhydride (PEGNB-CA, or PEGNB_{CA}). The tissue adhesive moiety may include, but is not limited to, catechol, hydrazide, fibrin glue, fibrinogen glue, a hydrogel tissue glue, a cyanoacrylate, chondroitin sulfate aldehyde, ketone, a derivative of any of these, or a combination of two or more of these.

In embodiments, the composition may further include biological material. In embodiments, the biological material may include a plurality of cells. In embodiments, the composition may comprise a single cell. In embodiments, the cell or plurality of cells may be include, are is not limited to the following cell types: a Brunner's gland cell, an insulated goblet cell, a foveolar cell, a chief cell, a parietal cell, a pancreative cell, a paneth cell, a type II pneumocyte cell, a club cell, a type I pneumocyte cell, a gall bladder epithelial cell, an intercalated duct cell, an intestinal brush border cell, an enteroendocrine cell (such as a K cell, an L cell, an I cell, a g cell, an enterochromaffin cell, an enterochromaffin-like cell, an N cell, an S cell, a D cell, and a Mo cell), a thyroid glad cell (such as thyroid epithelial cell and a parafollicular cell), a parathyroid glad cell (such as a parathyroid chief cell and an oxyphil cell), a pancreatic islet cell (such as an alpha cell, a beta cell, a delta cell, an epsilon cell, and a pancreatic polypeptide (PP) cell), a salivary gland mucous cell, a salivary gland serous cell, a von Ebner's gland cell, a mammary gland cell, a lacrimal gland cell, a ceruminous gland cell, an eccrine sweat gland dark cell, an eccrine sweat gland clear cell, an apocrine sweat gland cell, a gland of Moll cell, a sebaceous gland cell, a Bowman's gland cell, an anterior or intermediate pituitary cell (such as corticotropes, gonadotropes, lactotropes, melanotropes, somatotropes, and thyrotropes), a magnocellular neurosecretory cell, a parvocellular neurosecretory cell, a chromaffin cell, a keratinocyte, an epidermal basal cell, a melanocyte, a trichocyte (such as a medullary hair shaft cell, a cortical hair shaft cell, a cuticular hair shaft cell, a Huxley's layer hair root sheath cell, a Henle's layer hair root sheath cell, an outer root sheath hair cell), a surface epithelial cell (such as of the cornea, tongue, mouth, nasal cavity, etc.), a basal cell (such as of the cornea, tongue, mouth, nasal cavity, etc.), an intercalated duct cell, a striated duct cell, a lactiferous duct cell, an ameloblast, an odontoblast, a cementoblast, a neuron, an auditory inner hair cell, an auditory outer hair cell, a basal cell of olfactory epithelium, a cold-sensitive primary sensory neuron, a heat-sensitive primary sensory neuron, a Merkel cell of epidermis, an olfactory receptor neuron, a pain-sensitive primary sensory neuron, a photoreceptor cell (such as rod cells, blue-sensitive cone cells, green-sensitive cone cells, and red-sensitive cone cells), a proprioceptive primary sensory neuron, a touch-sensitive primary sensory neuron, a chemoreceptor glomus cell, an outer hair cell of vestibular system, and inner hair cell of vestibular system, a taste receptor cell, a cholinergic neuron, an adrenergic neuron, a peptidergic neuron, an inner pillar cell of organ of Corti, an outer pillar cell of organ of Corti, a border cell of organ of Corti, a Henson's cell of organ of Corti, a vestibular apparatus supporting cell, a tast bud supporting cell, an olfactory epithelium supporting cell, an olfactory ensheathing cell, a Schwann cell, a Satellite glial cell, an enteric glial cell, an interneuron (such as basket cells, cartwheel cells, stellate cells, golgi cells, granule cells, lugaro cells, unipolar brush cells, matinotti cells, chandelier cells, Cajal-Retzius cells, double-bouquet cells, neurogliaform cells, retina horizontal cells, amacrine cells, starburst amacrine cells, spinal interneurons, and Renshaw cells), a principal neuron (such as spindle neurons, fork neurons, pyramidal cells, place cells, grid cells, speed cells, head direction cells, Betz cells, stellate cells, boundary cells, bushy cells, Purkinje cells, and medium spiny neurons), an astrocyte, an oligodendrocyte, an ependymal cell, a tanycyte, a pituicyte, an anterior lens epithelial cell, a crystalline-containing lens fiber cell, an adipocyte (such as white fat cells and brown fat cells), a liver lipocyte, a cell of the adrenal cortex, a theca interna cell, a corpus luteum cell, a granulosa lutein cell, a theca lutein cell, a Leydig cell, a seminal vesicle cell, a prostate gland cell, a bulbourethral gland cell, a Bartholin's gland cell, a gland of littre cell, an uterus endometrium cell, a juxtaglomerular cell, a macula densa cell, a peripolar cell, a mesangial cell, a parietal epithelial cell, a podocyte, a proximal tubule brush border cell, a loop of Henle thin segment cell, a kidney distal tubule cell, a kidney collecting duct cell (principal and intercalated), a transitional epithelium cell, a duct cell, an efferent ducts cell, an epididymal principal cell, an epididymal basal cell, a circulatory system endothelial cell, a planum semilunatum epithelial cell, an organ of Corti interdental epithelial cell, a loose connective tissue fibroblast, a corneal fibroblast, a corneal keratocyte, a tendon fibroblast, a bone marrow reticular tissue fibroblast, a nonepithelial fibroblast, a pericyte, a hepatic stellate cell, a nucleus pulposus cell, a hyaline cartilage chondrocyte, a fibrocartilage chondrocyte, an elastic cartilage chondrocyte, an osteoblast, an osteocyte, an osteoprogenitor cell, a hyalocyte of vitreous body, a stellate cell of perilymphatic space, a pancreatic stellate cell, a skeletal muscle cell (such as red skeletal muscle cells, white skeletal muscle cells, intermediate skeletal muscle cells, nuclear bag cells, nuclear chain cells, and myosatellite cells), a cardiac muscle cell (such as SA mode cells and Purkinje fiber cells), a smooth muscle cell, a myoepithial cell, an erythrocyte, a megakaryocyte, a platelet, a monocyte, a connective tissue macrophage, an epidermal Langerhans cell, an osteoclast, a dendritic cell, a microglial cell, a neutrophil granulocyte, an eosinophil granulocyte, a basophil granulocyte, a mast cell, a helper T cell, a regulatory T cell, a cytotoxic T cell, a natural killer T cell (NKT cell), a B cell, a plasma cell, a natural killer cell (NK cell), a hematopoietic stem cell, an oogonium, a spermatid, a spermatocyte, a spermatogonium cell, a spermatozoon, a granulosa cell, a Sertoli cell, an epithelial reticular cell, and an interstitial kidney cell.

In some embodiments, the composition may further include at least one active pharmaceutical ingredient, also referred to as a “drug” or an “active ingredient.” As used herein, the term “active pharmaceutical ingredient” refers to a therapeutically active compound, as well as any prodrugs thereof and pharmaceutically acceptable salts, hydrates, and solvates of the compound and the prodrugs. As used herein, the term “pharmaceutically acceptable,” with respect to salts, hydrates, and solvates, refers to forms of the active pharmaceutical ingredient which are not deleterious to a patient and which are compatible with other ingredients, active ingredients, salts, or components. “Pharmaceutically acceptable” includes “veterinarily acceptable,” and thus includes both human and non-human mammal applications independently. In some embodiments, the composition may include both a drug and a cell. In embodiments, the composition may include both a plurality of drugs and a plurality of cells, including fragments of biological tissue. The hydrogel may be used to encapsulate the drugs, cells, or a combination of drugs and cells, and then be used in the wound dressings described herein. However, in other embodiments, the hydrogel may be used to encapsulate the drug, and the encapsulated drug may be applied to a subject in some mode other than the wound dressing, such as a component of a salve, gel, or ointment, for example.

In embodiments, the method for making a composition comprising a hydrogel includes adding a drug to the composition. Further, in the same or different embodiments, the method for making a composition comprising a hydrogel includes adding at least one cell to the composition. That is, in embodiments, the method for making a composition comprising a hydrogel includes adding a drug alone, a cell alone, or both a drug and a cell. In embodiments, the cell is a pluripotent stem cell. In embodiments, the pluripotent stem cell is from a mammal. In embodiments, the pluripotent stem cell is from a human. As used herein, the term “stem cell” refers to a cell from which other types of cells develop. Stem cells are generally broadly classified into one of three groups: embryonic stem cells, adult stem cells, and induced pluripotent stem cells. Embryonic stem cells are capable of differentiating into any cell type and are thus pluripotent. Adult stem cells may differentiate into one of several cell types, but not all cell types, and are thus multipotent. Induced pluripotent stem cells are stem cells that have been created by inducing adult cells to revert to stem cells. Induced pluripotent stem cells created in this manner may then differentiate into any cell type. Thus, as used herein, the term “pluripotent stem cell” may refer to either embryonic stem cells, induced pluripotent stem cell, or both.

In another aspect of the disclosure, there is provided a method for treating a wound in a subject in need thereof. The method includes forming the composition comprising the hydrogel precursor, applying the composition to the wound, and allowing the tetrazine amine of the second reaction product to react with the first reaction product to form the hydrogel. The reaction forms the hydrogel. In another embodiment, a method for using a wound dressing comprising a hydrogel formed from the hydrogel precursor may include allowing the tetrazine amine of the second reaction product to react with the first reaction product to form the hydrogel; applying the hydrogel to a wound dressing precursor, thereby forming the wound dressing; and applying the wound dressing to a wound.

The thus prepared hydrogel may be used in the form of a wound dressing, such as a bandage for example, or as a tissue sealant. Therefore, the hydrogel may be formed prior to applying the composition to the subject or after applying the composition to the subject by applying each of the reaction products to the patient sequentially. For instance, the composition could be applied to the subject in its hydrogel state or the reaction product of the hydroxyl-terminated first multi-arm polyethylene glycol and the carbic anhydride or the reaction product of the hydroxyl-terminated first multi-arm polyethylene glycol and the norbornene acid could be applied followed by the reaction product of the second multi-arm polyethylene glycol comprising the amido succinic acid and the tetrazine amine. Once both are applied, the inverse electron demand Diels-Alder (iEDDA) reaction will occur.

In embodiments, a method for manufacturing a wound dressing may include forming the composition comprising the hydrogel precursor described above; allowing the tetrazine amine of the second reaction product to react with the first reaction product to form the hydrogel, thereby forming a hydrogel; and applying the hydrogel to a support, thereby forming the wound dressing. Supports may include, without limitation, cloth, polymer substrates, collagen, fibrin, fibrinogen, heparin, hyaluronic acid (HA), gelatin, fibronectin, chitosan, dextran, glycosaminoglycans (GAGs), cellulose, alginate, as well as synthetic polymers, including polytetrafluoroethylene (PTFE), poly lactic-co-glycolic acid (PLGA), polyurethane, polyvinyl alcohol (PVA), polyvinylpyrrolidone, polystyrene, or a combination of two or more of these. Small molecules/proteins may also be present, such as factor XIII, thrombin, calcium chloride, bovine serum albumin, polyphenols, zinc oxide (ZnO) nanoparticles, nitrofurazone, dibutyryl cyclic adenosine monophosphate, or a combination of two or more of these.

In embodiments, a method for encapsulating a drug, a cell, or both a drug and a cell includes forming any embodiment of the composition comprising the hydrogel precursor; mixing the drug, the cell, or both the drug and the cell, with the composition; and allowing the tetrazine amine of the second reaction product to react with the first reaction product to form the hydrogel, thereby encapsulating the drug, the cell, or both the drug and the cell. Such drug or cellular encapsulation may be beneficial in the delivery of drugs to a subject when the drugs would otherwise be metabolized or rejected within the subjected. Likewise, such encapsulated cells may be useful for delivering cellular materials for genetic therapy, organ generation, or tissue repair. Additional possible encapsulants include, but are not limited to, vitamin A, vitamin C, vitamin E, zinc and copper ions, growth factors, including but not limited to fibroblast growth factor (FGF) and endothelial growth factor (EGF), antibiotics, endothelial cells, fibroblasts, thrombocytes, mesenchymal stem cells, other stem cells, and a combination of two or more of these.

In addition to the aspects and embodiments described and provided elsewhere in the present disclosure, the following non-limiting list of embodiments are also contemplated:
1. A composition comprising a hydrogel precursor, the hydrogel precursor comprising:
   a first reaction product of a hydroxyl-terminated first multi-arm polyethylene glycol and a compound selected from the group consisting of a carbic anhydride, a norbornene acid, and a combination thereof; and
   a second reaction product of a tetrazine amine and a second multi-arm polyethylene glycol comprising an amido succinic acid moiety.
2. The composition of clause 1, wherein the tetrazine amine comprises a methyltetrazine amine.
3. The composition of clause 1 or clause 2, further comprising a tissue adhesive moiety selected from the group consisting of catechol, hydrazide, and derivatives thereof, or a combination of two or more thereof.
4. The composition of any one preceding clause, further comprising a cell.
5. The composition of any one preceding clause, further comprising an active pharmaceutical ingredient.
6. The composition of any one preceding clause, wherein the composition produces a hydrogel having a shear modulus from 1 kPa to 100 kPa.
7. The composition of any one of clauses 1-5, wherein the composition produces a hydrogel having a G′ from 1 kPa to 100 kPa.
8. The composition of any one of clauses 1-5, wherein the composition produces a hydrogel having a hydrolytic degradation half life from 5 days to 75 days.
9 The composition of any one of clauses 1-5, wherein the composition produces a hydrogel having a hydrolytic degradation half life from 5 days to 20 days.
10. The composition of any one of clauses 1-5, wherein the composition produces a hydrogel having a hydrolytic degradation half life from 50 days to 70 days.
11. The composition of any one preceding clause, wherein the first multi-arm polyethylene glycol comprises eight arms.
12. The composition of any one preceding clause, wherein the second multi-arm polyethylene glycol comprises four arms.
13. The composition of clause 12, wherein the four arms are conjugated to an unsubstituted tetrazine amine.
14. The composition of clause 12, wherein three of the four arms are conjugated to an unsubstituted tetrazine amine and one of the four arms is conjugated to a methyltetrazine amine.
15. The composition of clause 12, wherein two of the four arms are conjugated to an unsubstituted tetrazine amine and two of the four arms are conjugated to a methyltetrazine amine.
16. The composition of clause 12, wherein one of the four arms is conjugated to an unsubstituted tetrazine amine and three of the four arms are conjugated to a methyltetrazine amine.
17. The composition of clause 12, wherein the four arms are conjugated to a methyltetrazine amine.
18. A wound dressing comprising:
   a hydrogel formed from a hydrogel precursor, the hydrogel precursor comprising:
      a first reaction product of a hydroxyl-terminated first multi-arm polyethylene glycol and a compound selected from the group consisting of a carbic anhydride, a norbornene acid, and a combination thereof; and
      a second reaction product of a tetrazine amine and a second multi-arm polyethylene glycol comprising an amido succinic acid moiety.
19. The wound dressing of clause 18, wherein the tetrazine amine comprises a methyltetrazine amine.
20. The composition of clause 18 or clause 19, further comprising a tissue adhesive moiety selected from the group consisting of catechol, hydrazide, and derivatives thereof, or a combination of two or more thereof.
21. The wound dressing of any one of clauses 18-20, wherein the hydrogel has a shear modulus from 1 kPa to 100 kPa.
22. The wound dressing of any one of clauses 18-21, wherein the hydrogel has a G′ from 1 kPa to 100 kPa.
23. The wound dressing of any one of clauses 17-22, wherein the hydrogel has a hydrolytic degradation half life from 5 days to 75 days.
24. The wound dressing of any one of clauses 18-23, wherein the hydrogel has a hydrolytic degradation half life from 5 days to 20 days.
25. The wound dressing of any one of clauses 18-24, wherein the hydrogel has a hydrolytic degradation half life from 50 days to 70 days.
26. The wound dressing of any one of clauses 18-25, wherein the first multi-arm polyethylene glycol comprises eight arms.
27. The wound dressing of any one of clauses 18-26, wherein the second multi-arm polyethylene glycol comprises four arms.
28. The wound dressing of clause 27, wherein the four arms are conjugated to an unsubstituted tetrazine amine.
29. The wound dressing of clause 27, wherein three of the four arms are conjugated to an unsubstituted tetrazine amine and one of the four arms is conjugated to a methyltetrazine amine.
30. The wound dressing of clause 27, wherein two of the four arms are conjugated to an unsubstituted tetrazine amine and two of the four arms are conjugated to a methyltetrazine amine.
31. The wound dressing of clause 27, wherein one of the four arms is conjugated to an unsubstituted tetrazine amine and three of the four arms are conjugated to a methyltetrazine amine.
32. The wound dressing of clause 27, wherein the four arms are conjugated to a methyltetrazine amine.
33. A method for manufacturing a wound dressing comprising:
   forming a composition comprising a hydrogel precursor, the hydrogel precursor comprising
      a first reaction product of a hydroxyl-terminated first multi-arm polyethylene glycol and a compound selected from the group consisting of a carbic anhydride, a norbornene acid, and a combination thereof; and
      a second reaction product of a tetrazine amine and a second multi-arm polyethylene glycol comprising an amido succinic acid moiety;
   allowing the tetrazine amine of the second reaction product to react with the first reaction product to form the hydrogel, thereby forming a hydrogel; and
   applying the hydrogel to a support, thereby forming the wound dressing.
34. The method of clause 33, further comprising
   applying the composition comprising the hydrogel precursor to a wound prior to allowing the tetrazine amine of the second reaction product to react with the first reaction product to form the hydrogel; and
   covering the composition with the support.
35. A method for using a wound dressing comprising a hydrogel formed from a hydrogel precursor, the hydrogel precursor comprising:
   a first reaction product of a hydroxyl-terminated first multi-arm polyethylene glycol and a compound selected from the group consisting of a carbic anhydride, a norbornene acid, and a combination thereof; and
   a second reaction product of a tetrazine amine and a second multi-arm polyethylene glycol comprising an amido succinic acid moiety;
the method comprising
   allowing the tetrazine amine of the second reaction product to react with the first reaction product to form the hydrogel;
   applying the hydrogel to a support, thereby forming the wound dressing; and
   applying the wound dressing to a wound.
36. A method for treating a wound in a subject in need thereof, the method comprising:
   forming the composition of any one of clauses 1-17;
   applying the composition to the wound; and
   allowing the tetrazine amine of the second reaction product to react with the first reaction product to form the hydrogel, thereby treating the wound.
37. The method of clause 36, wherein the subject is a human.
38. The method of clause 36 or clause 37, wherein applying the composition to the wound comprises applying the composition over the wound as a wound dressing.
39. The method of any one of clauses 36-38, wherein applying the composition to the wound comprises applying the composition within the wound as a tissue sealant.
40. A method for encapsulating a drug, a cell, or both a drug and a cell, the method comprising:
   forming the composition of any one of clauses 1-17;
   mixing the drug, the cell, or both the drug and the cell, with the composition; and
   allowing the tetrazine amine of the second reaction product to react with the first reaction product to form the hydrogel, thereby encapsulating the drug, the cell, or both the drug and the cell.

### Examples

Examples related to the present disclosure are described below. In most cases, alternative techniques can be used. The examples are intended to be illustrative and are not limiting or restrictive of the scope of the invention as set forth in the claims.

### Materials and Methods

Hydroxyl-terminated 8-arm PEG (20 kDa) and 4-arm PEG-amino succinic acid (PEG-ASA) (10 kDa) were purchased from JenKem Technology USA and Laysan Bio Inc., respectively. Carbic anhydride, pyridine, dichloromethane (DCM), and 1-(3-Dimethylaminopropyl)-3-ethylcarbodiimide hydrochloride (EDC) were all purchased from Thermo Scientific. 5-Norbornene-2-carboxylic acid, tetrahydrofuran (THF), 4-dimethylaminopyridine (DMAP), and N,N′-dicyclohexylcarbodiimide (DCC) were obtained from Sigma-Aldrich. Tetrazine amine and methyltetrazine amine were purchased from Click Chemistry Tools. N-hydroxylsuccinimide (NHS) and N,N-diisopropylethylamine (DIEA) were obtained from Tokyo Chemical Industry (TCI). N,N-dimethylformamide (DMF), 1-[Bis(dimethylamino)methylene]-1H-1,2,3-triazolo[4,5-b]pyridinium-3-oxidehexafluorophosphate (HATU), and cold soluble gelatin were purchased from Alfa Aesar, AnaSpec, and Modernist Pantry, respectively. Calcein-AM and ethidium homodimer stains were obtained from biotium. F-actin stain was purchased from Cytoskeleton, Inc.

### Macromer Synthesis and Purification

Synthesis of PEGNB and PEGNB_{CA}: 8-arm PEG-norbornene (PEGNB) was synthesized according to an established protocol. Briefly, 10 parts of norbornene acid was reacted with 5 parts of DCC in DCM to form norbornene anhydride with dicyclohexylurea as a byproduct. Dicyclohexylurea was removed through vacuum filtration with filter paper (size 52). Norbornene anhydride was then added dropwise into a flask containing hydroxyl-terminated 8-arm PEG (20 kDa), pyridine (10-fold to —OH), and DMAP (1-fold to —OH). All reactions occurred under nitrogen and the product was precipitated with diethyl ether and filtered using a fritted glass funnel. The PEGNB product was redissolved in double distilledwater (DDH₂O). 8-arm PEG-norbornene-carbic anhydride (PEGNB_{CA}) was synthesized using a published protocol. Briefly, hydroxyl-terminated 8-arm PEG (20 kDa) was reacted with 5-fold carbic anhydride and 0.5-fold DMAP in THF at 60° C. for 12 hours. Following 12 hours, a second portion of 5-fold carbic anhydride and 0.5-fold DMAP was added and proceed to react for another 24 hours. The PEGNB_{CA} product was precipitated with diethyl ether and redissolved in DDH₂O.

Synthesis of PEGTz, PEGmTz, and PEG-mTz/Tz: Tetrazine amine and/or methyltetrazine amine was conjugated onto 4-arm PEG-ASA (10 kDa) following a previous protocol. Briefly, PEG-ASA and 5-fold HATU was dissolved in DMF and allowed to react in order to form an active ester. Subsequently, either 1.2-fold tetrazine amine or methyltetrazine amine and 5-fold DIEA was added and allowed to react for 16 hours at room temperature. Similar to PEG-Tz and PEG-mTz syntheses, PEG-mTz/Tz ratios were obtained by one-pot reaction of various molar ratios of methyltetrazine amine and tetrazine amine onto 4-arm PEG-ASA. The feed molar ratios between methyltetrazine amine and tetrazine amine that were reacted are 1:3, 1:1, and 3:1.

Gel-mTz/Tz synthesis: Cold soluble gelatin (0.5 g), EDC (0.15 mmol), and NHS (0.15 mmol) were first dissolved in DDH₂O and allowed to react for 30 minutes. Tetrazine-amine (0.075 mmol) and methyltetrazine amine (0.075 mmol) was added to the reaction flask and was reacted overnight. All synthesized macromers were dialyzed with SpectraPor® regenerated cellulose dialysis membrane with molecular weight cut off (MWCO) of 3.5 kDa for 3 days and then lyophilized using a freeze dryer.

### Macromer Characterization

Substitution of norbornene derived from either carbic anhydride or 5-norbornene-2-carboxylic acid onto 8-arm PEG was determined using ¹H NMR (deuterium oxide, 500 MHz, Bruker Advance 500) by obtaining the integral peaks of the protons on the PEG-backbone to the alkene protons on the norbornene group. Substitution of tetrazine and/or methyltetrazine onto PEG-ASA and cold soluble gelatin was derived using ultraviolet-visible (UV-vis) spectroscopy against a standard curve (1 mg/mL to 0.015 mg/mL) of the respected ratio of free tetrazine amine and/or methyl-tetrazine amine. The actual ratio of tetrazine and methyltetrazine conjugated onto PEG-ASA was determined by comparing the integral peaks of the protons on the benzyl ring to the integral peaks on the methyl group of methyltetrazine.

### Hydrogel Crosslinking and Degradation

PEGNB and PEGNB_{CA} was crosslinked with PEGTz, PEGmTz, or PEG-mTz/Tz with different stoichiometric ratios of [Tz]: [Nb] (i.e., R=0.5, 1.0, and 2.0) and different macromer concentrations (i.e., 1.75 wt %, 2.5 wt %, and 4 wt %). To prepare hydrogels, 45 μL of polymer precursor solution was injected between two glass slides separated by 1 mm Teflon spacers. The slides containing the hydrogel precursor solution were then placed into a sealed container and allowed to react for 16 hours at room temperature. After 16 hours, the hydrogels were swelled in pH 7.4 phosphate buffer saline (PBS). Using an Anton-Paar MCR102 rheometer fitted with an 8 mm diameter parallel geometry plate, elastic (G′) and viscous (G″) moduli of the fabricated hydrogels were evaluated through strain sweep tests operating at 0.1% to 5% strain and 1 Hz oscillation frequency. Gelation kinetics was determined using in situ rheology performed with a 25 mm diameter parallel geometry plate. Hydrogel precursor solution was mixed briefly then 200 μL of solution was dispensed on the rheology stage. The plate was lowered to 0.2 mm and time sweep was conducted at 1% strain and 1 Hz frequency over 1 hour. In situ rheology was performed either at room temperature or 37° C.

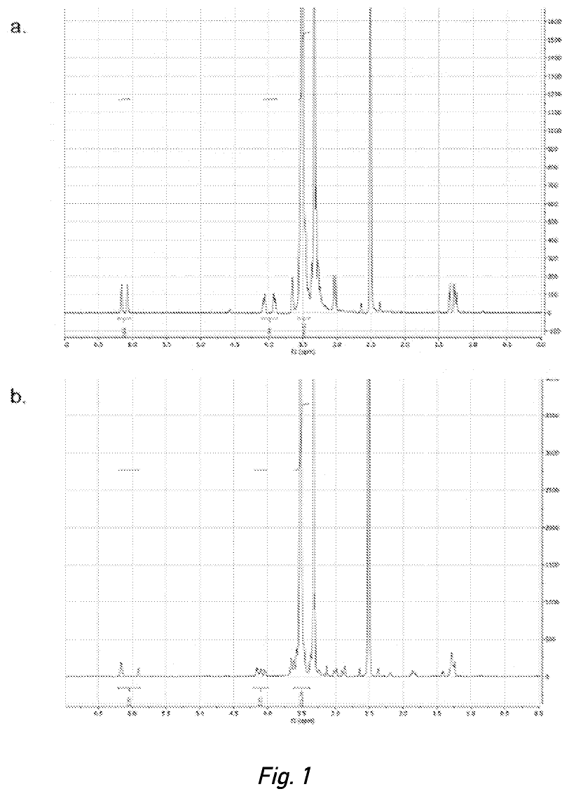

Norbornene-functionalized PEG can be synthesized by reacting hydroxyl-terminated PEG with either 5-norbornene-2-carboxylic acid (NB-acid) or carbic anhydride (CA), as shown in Scheme 2. We discovered that thiol-norbornene photo-click hydrogels crosslinked with the new PEGNB_{CA} degraded unexpectedly fast. The present Example sought to exploit the rapid degradation of hydrogels crosslinked by PEGNB_{CA} and establish the first hydrolytically degradable PEG-based iEDDA click hydrogels. The CA synthesis route produced higher degree of NB substitution efficiency (~91%, ca. 3.6 mM per wt % macromer), a value similar to that of PEGNB synthesized through conventional Steglich esterification between PEG and NB-acid (ca. 3.8 mM per wt %) (FIG. 1). To afford iEDDA click hydrogel crosslinking, mTz-amine or Tz-amine was conjugated to 4-arm PEG-ASA using standard carbodiimide chemistry with HTAU as the acid activator (Scheme 3). As expected, (m) Tz-modified PEGs were synthesized with high degree of substitution efficiency (~85% to 95%. Data not shown).

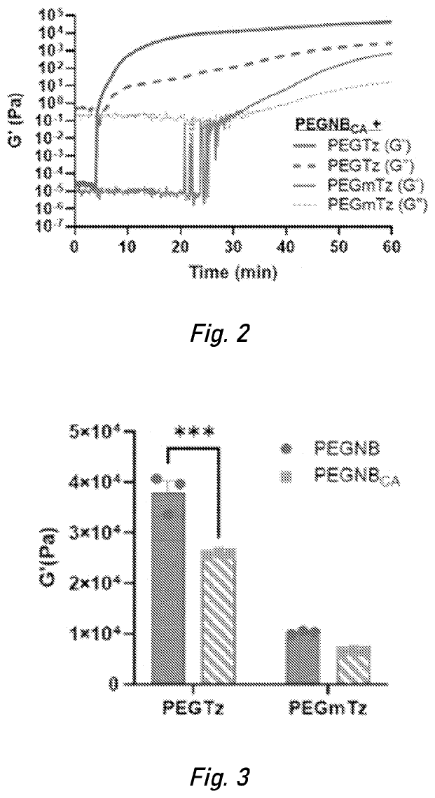

The crosslinking of iEDDA click hydrogel occurred upon simple mixing of PEGNB_{CA} and PEG (m) Tz (Scheme 1). In particular, when 2.5 wt % of PEGNB_{CA} was mixed with PEG (m) Tz at a stoichiometric ratio (R) of 1, G′/G″ crossover time for was ~4 minutes for PEGTz, whereas that for PEGmTz hydrogels was slower at ~30 minutes (FIG. 2). Initial shear moduli of PEGNB_{CA}/PEGTz hydrogels was (G′)-26 kPa, a value lower than that obtained from hydrogels crosslinked by conventional PEGNB (G′−38 kPa. FIG. 3). Compared with PEGNB, the lower crosslinking efficiency of hydrogels crosslinked by PEGNB_{CA} was similar to that observed in thiol-norbornene photocrosslinking. PEGNB hydrogels crosslinked with PEGmTz showed slightly higher but not statistically significant difference in G′ (−10 kPa) than those crosslinked by PEGNB_{CA} (G′-6.5 kPa. FIG. 3). After one day of swelling, the average G′ of PEGNB hydrogels crosslinked with PEGmTz increased to over 8 kPa; whereas, the average G′ of PEGNB hydrogels were essentially the same (data not shown).

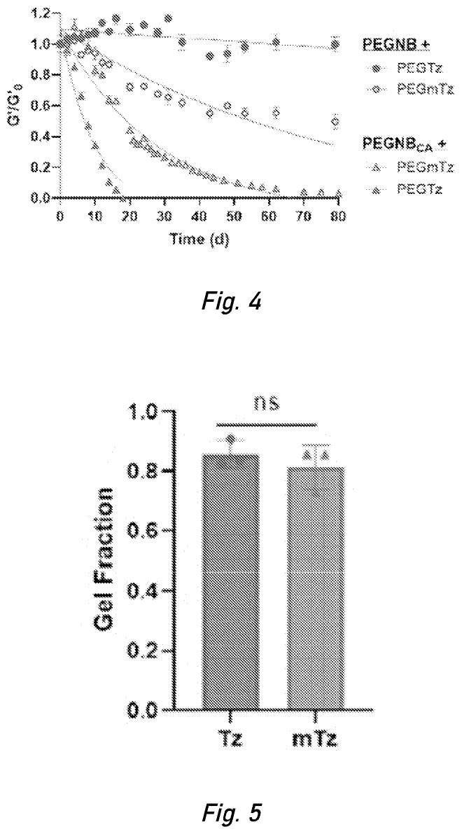

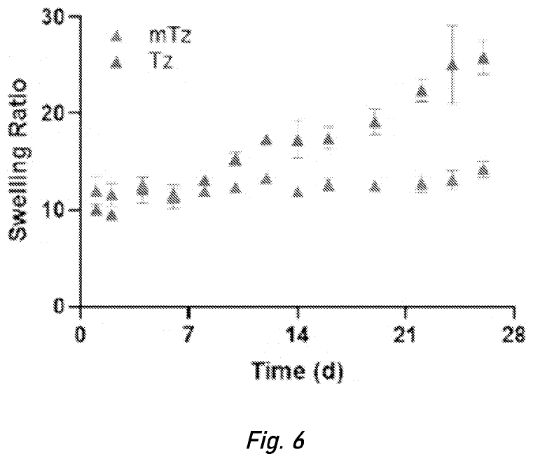

The degradation of synthetic PEG-based iEDDA-based NB-Tz click hydrogels was possible due to the presence of ester bond on PEGNB or PEGNB_{CA} (Scheme 1). Interestingly, iEDDA click hydrogels crosslinked by PEGNB and PEGTz showed little degradation over the course of 80 days (PEGNB+PEGTz, FIG. 4). On the other hand, PEGNB+PEGmTz iEDDA click hydrogels degraded slowly with a pseudo first-order hydrolysis rate constant (k_{hyd}) of ~0.0114 day-1 (Table 1, infra). As expected, iEDDA click hydrogels crosslinked by PEGNB_{CA} with either PEGTz or PEGmTz exhibited fast hydrolysis rate. Surprisingly, however, PEGNB_{CA}+PEGmTz iEDDA click hydrogels degraded much slower than PEGNB_{CA}+PEGTz hydrogels, with complete degradation occurring on day 80 and day 18 and k_{hyd} values of 0.0481 day-1 and 0.1021 day 1, respectively. Gel fractions were obtained to show a similar degree of crosslinking efficiency between PEGTz and PEGmTz with PEGNB_{CA} (FIG. 5). Hydrogel mass was tracked over time to determine swelling ratio and the mode of hydrogel degradation (e.g., surface erosion or bulk degradation (FIG. 6). Due to increased swelling ratio within the 28-day period for PEGNB_{CA}+PEGTz hydrogels, we reasoned that the hydrogels degraded following bulk degradation mechanism.

TABLE 1

| NB | M(Tz) | | | |
|---|---|---|---|---|
| Macromer | Macromer | k_{hyd} (d⁻¹) | Half Life (d) | R² |
| PEGNB | PEGTz | N/A | N/A | −0.35 |
| | PEGmTz | 0.0114 | 60.69 | 0.88 |
| PEGNB_{CA} | PEGTz | 0.1021 | 6.4788 | 0.86 |
| | PEGmTz | 0.0481 | 14.41 | 0.97 |

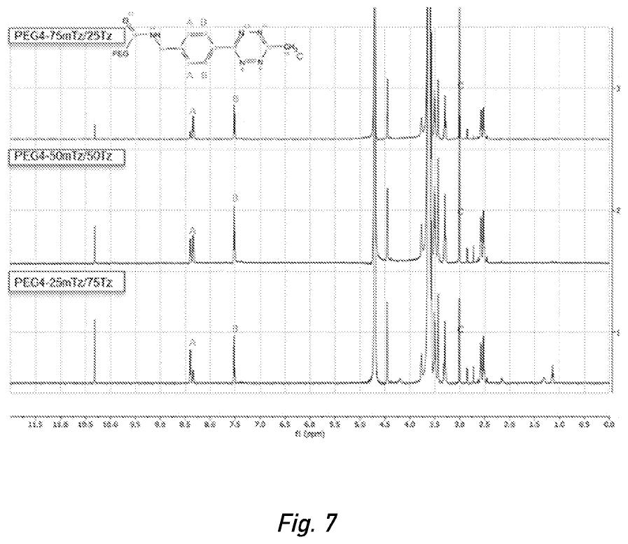

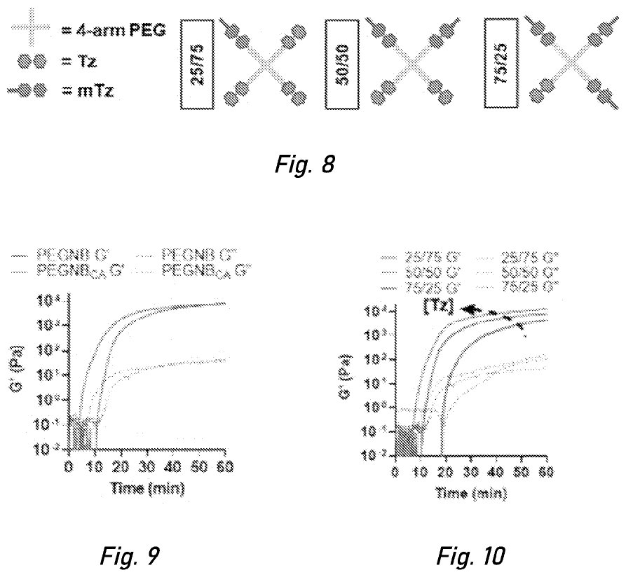

Encouraged by the discovery that using PEGTz or PEGmTz led to strikingly different hydrolytic degradation kinetics in PEGNB/PEGNB_{CA} based iEDDA click hydrogels, mTz/Tz dually modified PEG macromers were synthesized in an attempt to engineer degradation kinetics of PEG-based iEDDA click hydrogels. mTz/Tz dually modified PEGs were synthesized by controlling the feed ratios of methyltetrazine-amine to tetrazine-amine, yielding three sets of macromers with 75%-25%, 50%-50%, and 25%-75% (% represents molar feed ratio of mTz to Tz). The actual functional group ratios were determined by ¹H NMR spectra using the integral peaks from the protons on the methyl group to the protons on the benzene ring (FIGS. 7, 85%-15%, 69%-31%, and 35%-65%, respectively). The substitution efficiencies, as determined spectrophotometrically (at 523 nm), were ~93% (ca. 3.7 mM per wt %) for PEG-75mTz/25Tz and PEG-50 mTz/50 Tz, and ~85% (ca. 3.4 mM per wt %) for PEG-25Tz/75mTz. For simplicity, the mTz/Tz dual modified PEGs were denoted as PEG-75mTz/25Tz, PEG-50 mTz/50 mTz, and PEG-25mTz/75Tz, respectively (FIG. 8).

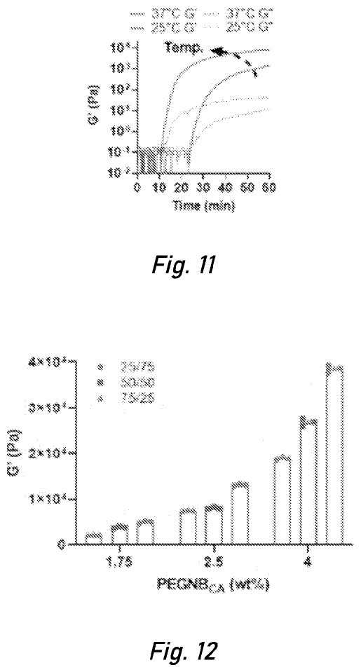

In situ rheology was performed to determine gelation point of PEGNB_{CA} compared to PEGNB crosslinked with PEGmTz/Tz crosslinker. The iEDDA click crosslinked PEGNB_{CA} hydrogels demonstrated slower gelation times with G′/G″ crossover at ~11 minutes compared to PEGNB hydrogels with crossover point at ~4 minutes (2.5 wt % PEGNB with PEG-50 mTz/Tz at R=1, 37° C. FIG. 9). Different gelation times were observed using the three mTz/Tz crosslinkers (FIG. 10). G′/G″ crossover time for 2.5 wt % PEGNB_{CA} crosslinked with PEG-25mTz/75Tz, PEG-50 mTz/50 Tz, and PEG-75mTz/25Tz (at R=1, 37° C.) was ~7 minutes, ~11 minutes, and ~18 minutes, respectively. Further, the iEDDA click crosslinked PEGNB_{CA} demonstrated temperature sensitive gelation behavior (FIG. 11). For 2.5 wt % PEGNB_{CA} hydrogels crosslinked with PEG-50 mTz/50 Tz (R=1), G′/G″ crossover time was ~24 minutes at 25° C. compared to ~11 minutes at 37° C.

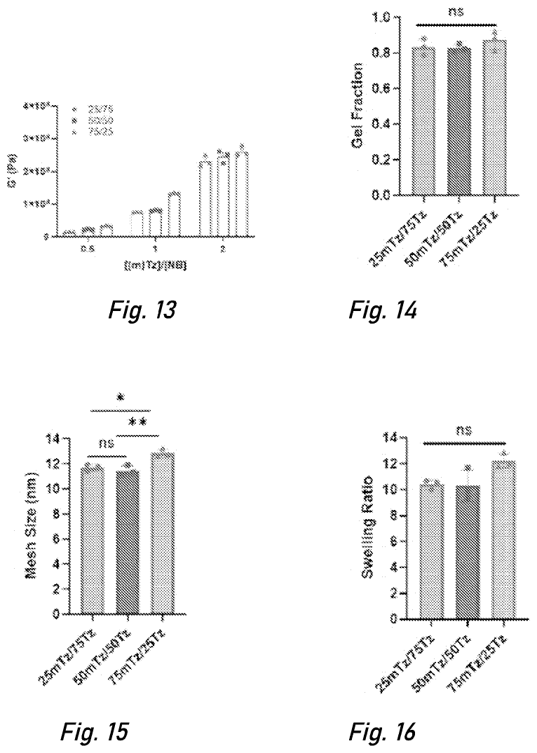

As with other click based hydrogels, initial G′ could be readily tuned by varying polymer weight percent and the ratio between tetrazine and norbornene (FIG. 12 and FIG. 13). At various polymer concentrations at a constant R, PEGNB_{CA} crosslinked with PEG-25mTz/75Tz had significantly higher elastic moduli compared to equivalent hydrogels crosslinked with PEG-50 mTz/50 Tz and PEG-75mTz/25Tz. The same trend was observed at different R ratios besides at R=2, where the initial G′ for all three macromers showed no statistically significant difference. Further, no statistical difference was observed in gel fraction and initial swelling ratio of PEGNB_{CA} crosslinked with the various PEG-mTz/Tz hydrogels (FIG. 14 and FIG. 15). However, for calculated mesh size, PEGNB_{CA} crosslinked with PEG-75mTz/25Tz hydrogels had a slightly larger mesh size compared to PEGNB_{CA} crosslinked with PEG-50 mTz/50 Tz and PEG-25mTz/75Tz (FIG. 16).

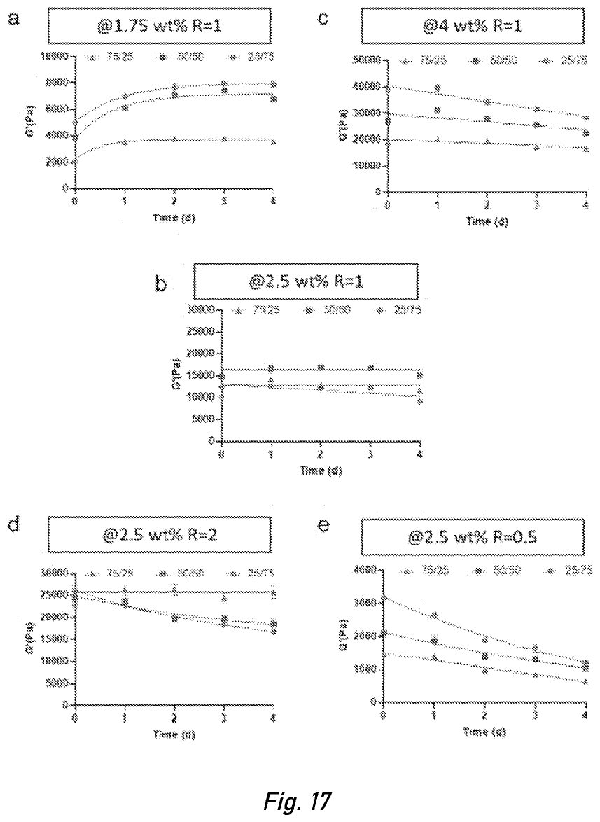

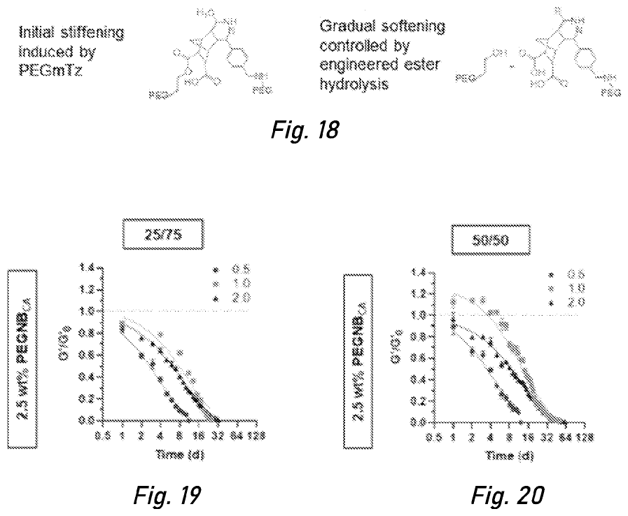

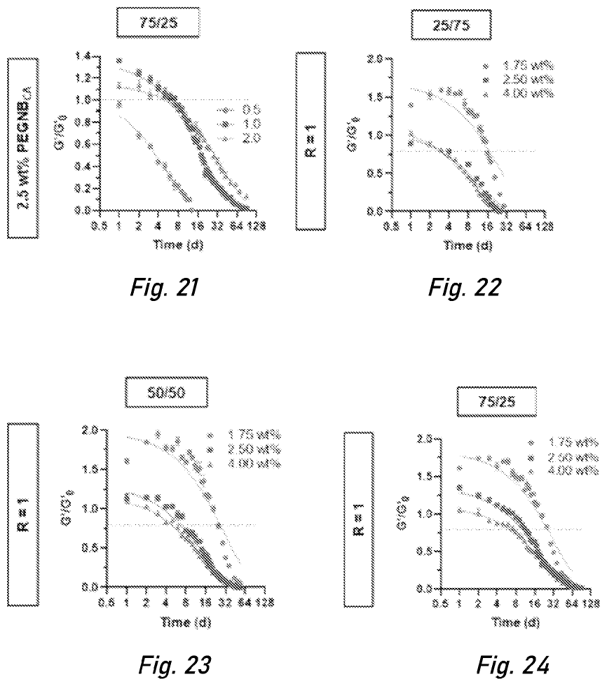

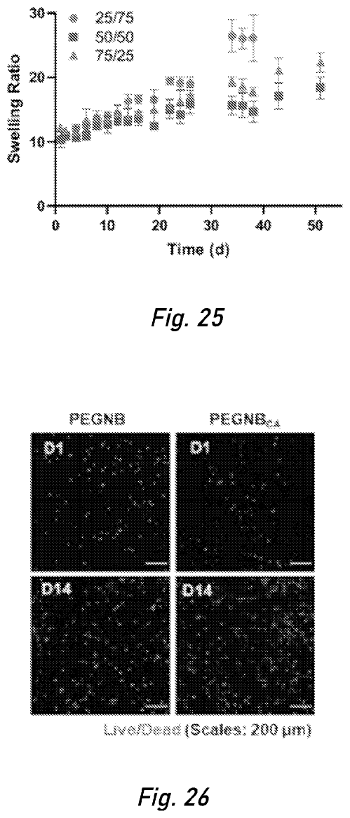

To assess hydrolytic degradation of PEGNB_{CA}-based iEDDA click hydrogels, the fabricated hydrogels were swelled in PBS pH 7.4 at 37° C. and elastic moduli was tracked over a 90-day period. At 1.75 wt % and R=1, the hydrogels exhibited significant stiffening during the first week (FIG. 17), followed by rapid degradation of hydrogels (FIG. 18) crosslinked with PEG-25mTz/75Tz over 30 days, while hydrogels crosslinked with PEG-50 mTz/50 Tz and PEG-75mTz/25Tz degraded much slowly and reached complete degradation after 60 days (Compare FIG. 19 and FIG. 22). At higher PEGNB_{CA} concentration (i.e., 2.5 wt % and 4 wt % at R=1), no stiffening was observed (FIG. 17) but the hydrogels crosslinked with the three different crosslinkers exhibited different degradation rates. For instance, at 4 wt % PEGNB_{CA} crosslinked with PEG-25mTz/75Tz, PEG-50 mTz/50 Tz, and PEG-75mTz/25Tz, hydrogels completely degraded within 28 days (FIG. 22), 55 days (FIG. 23), and 80 days (FIG. 24) with k_{hyd} values of 0.1186 day⁻¹, 0.0778 day⁻¹, and 0.0486 day⁻¹, respectively (Table 2, infra). Hydrolytic degradation of PEGNB_{CA}-based hydrogels was inversely proportional to the R ratio. For example, hydrogels crosslinked with PEG-75mTz/25Tz at R=0.5, 1, and 2 (FIG. 21) proceeded with k_{hyd} values of 0.1930 day⁻¹, 0.0532 day⁻¹, and 0.0281 day⁻¹, respectively. Similar trends were exhibited for PEG-25mTz/75Tz samples (FIG. 19) and for PEG-50 mTz/50 Tz (FIG. 20). Swelling ratio was tracked over time as another method for observing the rate of hydrolysis. Over a 50-day period, PEGNB_{CA} hydrogels crosslinked with PEG-50 mTz/50 Tz or PEG-75mTz/25Tz had a swelling ratio twice as large as its initial values, while PEGNB_{CA}+PEG-25mTz/75Tz hydrogels achieved maximum swelling ratio on day 38 (FIG. 25)

TABLE 2

| | | | |
|---|---|---|---|
| Sample | k_{hyd} (d⁻¹) | Half Life (d) | R² |

@2.5 wt % R = 0.5

| | | | |
|---|---|---|---|
| PEG-75mTz/25Tz | 0.193 | 5.18 | 0.96 |
| PEG-50mTz/50Tz | 0.203 | 4.926 | 0.95 |
| PEG-25mTz/75Tz | 0.262 | 3.817 | 0.98 |

@2.5 wt % R = 1

| | | | |
|---|---|---|---|
| PEG-75mTz/25Tz | 0.0532 | 13.02 | 0.98 |
| PEG-50mTz/50Tz | 0.0647 | 10.72 | 0.93 |
| PEG-25mTz/75Tz | 0.0907 | 7.646 | 0.94 |

@2.5 wt % R = 2

| | | | |
|---|---|---|---|
| PEG-75mTz/25Tz | 0.281 | 24.71 | 0.97 |
| PEG-50mTz/50Tz | 0.0817 | 8.484 | 0.98 |
| PEG-25mTz/75Tz | 0.1147 | 6.043 | 0.99 |

@1.75 wt % R = 1

| | | | |
|---|---|---|---|
| PEG-75mTz/25Tz | 0.0368 | 18.85 | 0.96 |
| PEG-50mTz/50Tz | 0.0414 | 16.73 | 0.94 |
| PEG-25mTz/75Tz | 0.0554 | 12.5 | 0.88 |

@4 wt % R = 1

| | | | |
|---|---|---|---|
| PEG-75mTz/25Tz | 0.0486 | 14.27 | 0.98 |
| PEG-50mTz/50Tz | 0.0778 | 8.913 | 0.99 |
| PEG-25mTz/75Tz | 0.1186 | 6.402 | 0.96 |

### Cell Encapsulation and Analysis

hMSC Culture: Human mesenchymal stem cells (hMSCs) were isolated from donor bone marrow (acquired from Lonza) and cultured in low glucose (1 g/L) Dulbecco's modified eagle medium (DMEM) supplemented with 10% fetal bovine serum (FBS), 1% antibiotic-antimycotics, and 1 ng/mL basic fibroblast growth factor (bFGF). Media was changed every 3-4 days. Once cell confluency reached ~80%, cells were passaged using trypsin. For this study, hMSCs had passage numbers between 4-6.

Cell Encapsulation: Detached single-cell hMSCs were encapsulated in 2.5 wt % PEG-NB or PEGNB_{CA} hydrogels crosslinked with PEG-25mTz/75Tz at R=0.5. To improve biocompatibility, 3 wt % Gel-50 mTz/50 Tz was reacted 1 hour before at 37° C. before gelation occurred. After 7 minutes of reacting at 37° C. for PEGNB_{CA} hydrogels and 2 minutes for PEGNB hydrogels, 25 μL of hydrogel precursor solution containing hMSCs were placed in open-tip 1 mL syringes. The cell-laden hydrogels were then allowed to react for another 20 minutes before being placed in cell culture media. As before, media changes occurred every 3-4 days.

Live/Dead and F-actin/DAPI staining: On day 1 and day 14, cell-laden hydrogels were washed with Dulbecco's phosphate-buffered saline (DPBS) for 5 minutes. Next, cell-laden hydrogels were incubated with 0.3 L/mL of Calcein AM and 0.26 μL/mL of ethidium homodimer for 1 hour at room temperature protected from light. After 1 hour, the cell-laden hydrogels were washed three times for 5 minutes using DPBS. On day 14, cell-laden hydrogels were washed twice for 5 minutes with DPBS and fixed with 4% paraformaldehyde for 45 minutes. Fixed cells within the hydrogel were then stained with 140 nM of F-actin (Acti-stain™ 555 Fluorescent Phalloidin, Cytoskeleton, Inc.) in the presence of 1% (v/v) bovine serum albumin (BSA) and 0.3% % (v/v) triton X100 at 4° C. for overnight. F-actin stained cells in the hydrogel were washed three times for 30 minutes with 1% (v/v) BSA and 0.3% (v/v) triton X100 and then counterstained with 4′,6-diamidino-2-phenylindole (DAPI) for 1 hour at room temperature. The stained cells in the hydrogel were washed with DPBS then imaged using confocal microscopy (Olympus Fluoview, FV1000). For analysis, a total of three hydrogels were imaged per condition with at least three random images per gel.

Image Analysis: ImageJ was used to quantify morphology of F-actin/DAPI stained hMSCs in PEGNB and PEGNB_{CA} hydrogels. Briefly, images were processed using fill holes and water-shed features. Subsequently, the analyze particle feature was applied with appropriate size threshold set and shape descriptor for each image. Circularity was calculated using the following equation:

$$Circularity = \frac{4 rc Area}{{perimeter}^{2}}$$

Aspect ratio was also calculated through ImageJ using the equation:

$$Aspect Ratio = \frac{{width}^{2}}{Area}$$

In vivo injection and histological evaluation: All animal studies were approved by the Indiana University Purdue University Indianapolis School of Science Institutional Animal Care and Use Committee (Approval number: SC303R). A total of six C57BL/6 mice were used. Under sterile conditions, 50 μL of 2.5 wt % PEGNB or PEGNB_{CA} crosslinked with PEG-25mTz/75Tz with R=0.8 and 3 wt % Gel-50 mTz/50 Tz was injected into the region of quadriceps muscle of the left hindlimb of each mouse. Body weight of the mice were measured weekly. After two weeks, mice were sacrificed and the quadriceps muscle form the left hindlimb was collected. The collected muscle tissue was fixed in formalin for one day and subsequently stored in 70% ethanol. Tissue specimens were embedded in paraffin, sectioned, and stained with hematoxylin and eosin (H&E), CD45 (Cell Signaling Technology, Rabbit mAb), and CD68 (Cell Signaling, Rabbit mAb).

Statistics: Data is presented as mean #SEM. When there are more than two conditions, one-way ANOVA or two-way ANOVA with Tukey multiple comparison test was used to determine statistical significance between groups when p<0.05. When there are only two conditions, unpaired t-test was used to determine statistical significance with p<0.05. Using GraphPad Prism 9, normalized elastic moduli data over time was fitted to a one phase decay. From here, k_{hydrolysis} constants and R² values were obtained.

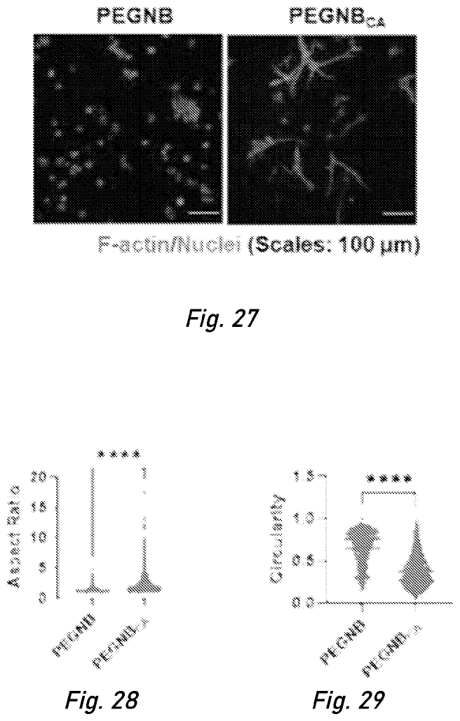

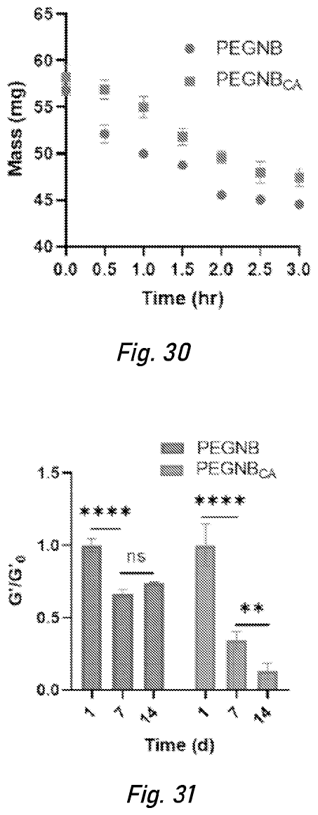

To test the cytocompatibility of the iEDDA click crosslinked PEGNB_{CA} hydrogels, hMSCs were encapsulated in 2.5 wt % PEGNB_{CA} or PEGNB hydrogels crosslinked by PEG-25mTz/75Tz with R=0.5. Additionally, gelatin functionalized with 50 mTz/50 Tz (3 wt %) was added to promote cell adhesion. Live/dead staining and confocal imaging demonstrated excellent cytocompatibility of both PEGNB_{CA} and PEGNB-based hydrogels, with both gels supported higher than 99% of viable cells one day post-encapsulation (FIG. 26). After 14 days of culture, hMSCs displayed an extensive spreading morphology only within PEGNB_{CA} hydrogels, whereas cells remained largely rounded in PEGNB hydrogels. The difference in morphology between the two gel formulations were evaluated using anti-F-actin and DAPI staining on day 14 (FIG. 27), which reaffirmed the extensive spreading morphology of hMSCs within iEDDA click crosslinked PEGNB_{CA} hydrogels. Image analysis demonstrated statistical difference in circularity and aspect ratio between the two conditions (FIG. 28 and FIG. 29). Mass of iEDDA click crosslinked PEGNB and PEGNB_{CA} hydrogels with gelatin were measured in the presence of collagenase to demonstrate both hydrogels were equally susceptible to proteolytic degradation (FIG. 30). Further, strain sweep tests were performed on day 1, day 7, and day 14 (FIG. 31). Initially, iEDDA click crosslinked PEGNB_{CA} hydrogels had an average shear modulus greater than 4 kPa. After 14 days, the average elastic modulus was ~0.5 kPa. Alternatively, iEDDA click crosslinked PEGNB hydrogels had an average shear modulus close to 3 kPa. On day 7 and day 14, the average shear moduli decreased compared to day 1 due to quick degradation of gelatin in nonsterile conditions causing it to be ~2 kPa. Without intending to be bound by any particular theory, since the two sets of hydrogels contained the same amount of gelatin, it is believed that the spreading morphology observed in PEGNB_{CA} hydrogels relative to PEGNB hydrogels may be attributed to accelerated hydrolysis.

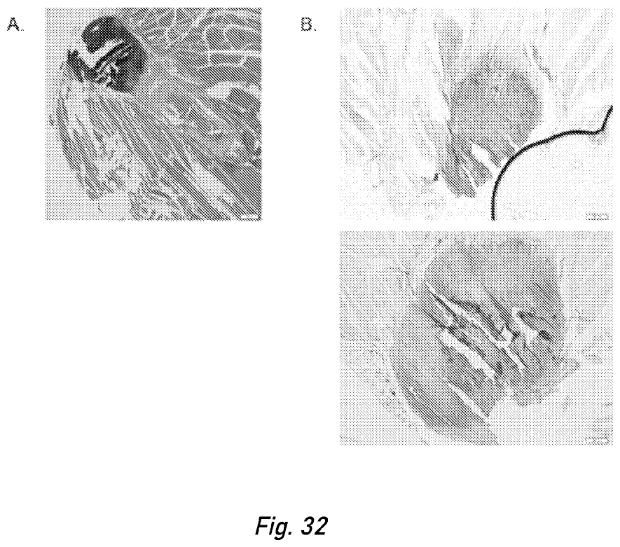

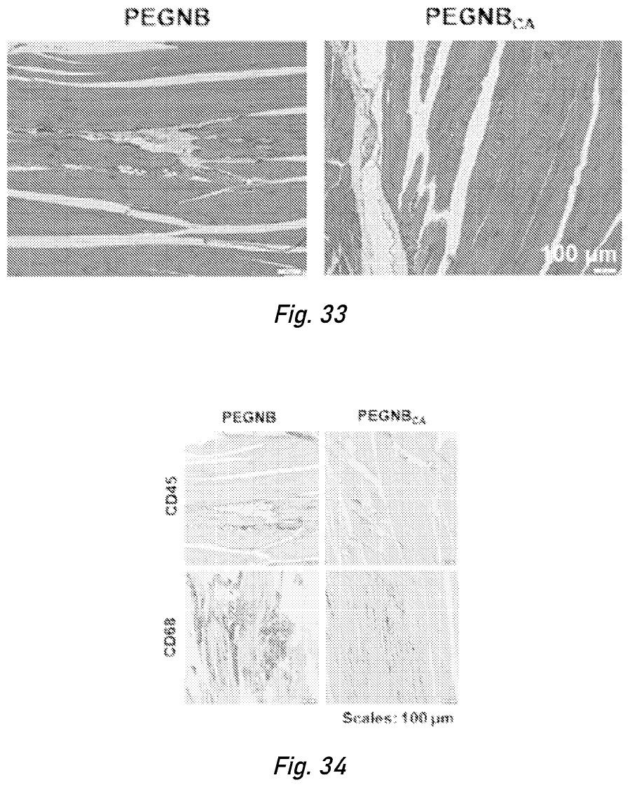

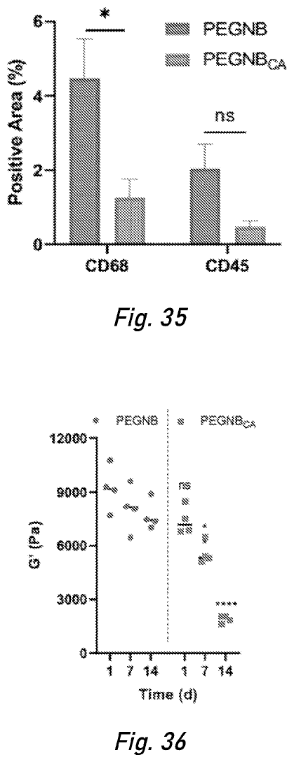

To test injectability and in vivo biocompatibility of the iEDDA click crosslinked PEGNB_{CA} hydrogels, 2.5 wt % PEGNB_{CA} or PEGNB crosslinked with PEG-25mTz/75Tz was injected subcutaneously into the left hindlimb of mice. After two weeks, mice were sacrificed and immunohistochemical staining (H&E, CD45, and CD68) was performed (FIG. 32). No remnants of PEGNB_{CA} hydrogels were present in all histology samples indicating complete degradation of the hydrogel before sample recovery. Gel fragments of PEGNB hydrogels were present in two of the three samples, indicating that the hydrogels were successfully injected and did not degrade completely. No noticeable difference in H&E staining of the tissue was observed between the two conditions as shown in the microscope images (FIG. 33). CD68 and CD45 staining of histology samples showed higher macrophage and lymphocyte activity, respectively, in PEGNB condition compared to PEGNB_{CA} (FIG. 34). Image analysis showed higher percent positive area of CD68 and CD45 images of histology samples with PEGNB hydrogel compared to PEGNB_{CA} (FIG. 35). In fact, CD68 percent positive area was statistically higher in histology samples with PEGNB hydrogel compared to PEGNB_{CA} hydrogel. Initial elastic moduli of injected iEDDA PEGNB or PEGNB_{CA} hydrogel was similar (FIG. 36). However, after 14 days, the average elastic moduli of PEGNB_{CA} hydrogels decreased to ~2 kPa, while PEGNB hydrogels had statistically significant higher average elastic modulus of ~7.5 kPa (FIG. 36).

### DISCUSSION

PEG-based hydrogels with engineered functionality and degradability are invaluable in tissue engineering and drug delivery applications. In particular, norbornene-functionalized PEG (PEGNB) is increasingly used in PEG-based hydrogel fabrication owing to its high cytocompatibility and dual reactivity towards thiol (in thiol-NB click reactions) and (m) Tz (NB-(m) Tz click reactions). Carbic anhydride (CA) was conjugated onto PEG terminal hydroxyl groups via esterification using DMAP as a catalyst. The CA underwent cyclic desymmetrization yielding PEGNB_{CA}, a functional macromer with norbornene group and a carboxylic acid that can be utilized for additional functionalization (Scheme 2). Successful synthesis of PEGNB_{CA} and PEGTz/PEGmTz permitted bio-orthogonal hydrogel crosslinking via iEDDA click reaction (Scheme 1). It was demonstrated that upon crosslinking into hydrogels, PEGNB_{CA}-based hydrogels displayed unexpected fast hydrolytic degradation kinetics, presumably a result of accelerated hydrolysis of the ester linkages caused by the neighboring carboxylic acid.

While both PEGNB and PEGNB_{CA} provided norbornene moiety for iEDDA click hydrogel crosslinking, the presence of carboxylic acid on PEGNB_{CA} appeared to slightly reduce iEDDA hydrogel crosslinking efficiency (FIG. 3). In addition to PEGNB and PEGNB_{CA}, two Tz-crosslinkers, PEGTz and PEGmTz, were synthesized and examined for hydrolytic stability of these four sets of iEDDA click hydrogels over 80 days (FIG. 4). PEGNB+PEGTz hydrogels exhibited exceptional hydrolytic stability with no noticeable degradation over 80 days. This was attributed to the relative stability of PEGNB-ester bonds formed by Steglich esterification, as well as the secondary non-covalent bonding between the NB-Tz adducts. Interestingly, iEDDA click hydrogels crosslinked by PEGmTz (PEGNB+PEGmTz group) underwent gradual stiffening (20% higher G′) and subsequently showed significant hydrolytic degradation in the first 45 days, followed by a less pronounced degradation afterward. The 20% stiffening was likely a result of slow reaction kinetics between NB and mTz. Following gelation, without intending to be bound by any particular theory, it is believed that the presence of additional methyl group on mTz disrupted the hydrogen bonding and T-T stacking between NB-Tz adducts, leading to noticeable hydrolytic degradation of PEGNB+PEGmTz hydrogels. The hydrolytic degradation of PEGNB_{CA}-based iEDDA click hydrogels were next examined, and it was found that PEGNB_{CA}+PEGTz hydrogels degraded much faster than PEGNB_{CA}+PEGmTz hydrogels. Through exponential pseudo-first order decay fitting of the shear moduli data over time, it was shown that the k_{hyd} for PEGNB_{CA}+PEGTz was 2.1 times faster than that in PEGNB_{CA}+PEGmTz group (Table 1, supra). While the methyl groups in mTz promoted degradation of PEGNB-based iEDDA click hydrogels (due to disruption of R-T stacking), it reduced the degradation rate of PEGNB_{CA}-based hydrogels, presumably because there was a lack of π-π stacking and PEGmTz was more hydrophobic than PEGTz. Nonetheless, the first PEG-based iEDDA click hydrogels with pre-engineered hydrolytic degradability are demonstrated herein.

To further explore the tunability of hydrolytically degradable iEDDA click hydrogels, dually modified mTz/Tz PEG was synthesized to control gelation and degradation kinetics. It is known that through the addition of electron withdrawing groups on the dienophile (e.g., norbornene) and electron donating groups on the diene (e.g., tetrazine) the reaction kinetics are slowed. Thus, PEGNB_{CA} iEDDA click crosslinked hydrogels may have slower gelation times compared to the equivalent PEGNB hydrogels (FIG. 8). Further, previous studies have shown that hydrogels crosslinked with mTz have slower gelation times compared to the same hydrogels crosslinked with Tz. In view of this. Tz and mTz were functionalized on the same polymer to allow for decreased gelation time by the fast-reacting tetrazine to form hydrogel initially then the mTz/NB reaction can occur within the hydrogel network over time. For all three different crosslinkers, quick gelation times occurred in less than 20 minutes (FIG. 9). Similar to previous studies, at higher temperatures, the gelation time is decreased in iEDDA click crosslinked PEGNB_{CA} hydrogels (FIG. 10), making it ideal for injectable applications. Compared to thiol-norbornene crosslinking, much lower macromer concentration is needed to achieve high elastic moduli (FIG. 11 and FIG. 12). Further, at low macromer concentration with R ratio equal to 1, increased shear moduli of iEDDA click crosslinked hydrogels were observed after day 0 indicating that the increase in stiffness is possibly from the slow reaction kinetics of mTz or from delayed supramolecular interactions from the tetrazine adducts. As a result, this hydrogel system will be useful in studying the effect of dynamic stiffening with subsequent softening without the need of external stimuli such as light or enzyme. Highly tunable degradation was achieved through the PEG-mTz/Tz ratio crosslinkers with higher ratio of mTz degrading slower across all different formulations (FIGS. 19-24). Further, all conditions adhered closely to pseudo-first order kinetics by having R² values greater than 0.93 excluding 1.75 wt % PEGNB_{CA} crosslinked with PEG25mTz75Tz at R=1 and displayed a wide range of degradation from 11 days to over 90 days.

Due to their prevalence in tissue engineering and mechanobiology, hMSCs were chosen to assess the cytocompatibility and morphology within the slow degrading PEGNB and fast degrading PEGNB_{CA} IEDDA click crosslinked hydrogels. Within a degrading matrix, hMSCs display a spreading morphology, while in a statically stiff matrix hMSCs maintain a round morphology. Similarly, hMSCs within the hydrolytically degradable PEGNB_{CA}-based iEDDA click hydrogels exhibited spreading morphology; whereas, hMSCs within the non-degradable hydrogels maintained a spherical morphology. Of note, in both hydrogels, mTz/Tz functionalized (50/50) gelatin was added to promote cell adhesion and protease labile sites. Due to the ease of functionalizing (m) Tz onto biopolymers (i.e. gelatin and hyaluronic acid), the system described herein should be highly adaptable for creating modular and biomimetic matrices.

The spontaneous reaction of Tz/NB iEDDA click chemistry is ideal for biomedical applications owing to its specific and high reactivity under ambient conditions without the need of external stimuli. In particular, this chemistry has been applied for in vivo applications including fluorescent imaging, ligation of biomolecules, and injectable, covalently crosslinked hydrogels. Due to the fast gelation time and predictable degradation, PEGNB_{CA} HEDDA click crosslinked hydrogels may be ideal for injectable hydrogel delivery. Due to the degrading matrix, a less degree of immune response compared to the statically stiff PEGNB hydrogels may be observed. Overall, PEGNB_{CA} HEDDA click crosslinked hydrogels produced less of an inflammatory response as indicated by CD45 and CD68 staining and subsequent image analysis (FIG. 34 and FIG. 35).

While embodiments have been disclosed hereinabove, the present invention is not limited to the disclosed embodiments. Instead, this application is intended to cover any variations, uses, or adaptations of the invention using its general principles. Further, this application is intended to cover such departures from the present disclosure as come within known or customary practice in the art to which this invention pertains and which fall within the limits of the appended claims.

1. A composition comprising a hydrogel precursor, the hydrogel precursor comprising:
a first reaction product of a hydroxyl-terminated first multi-arm polyethylene glycol and a compound selected from the group consisting of a carbic anhydride, a norbornene acid, and a combination thereof; and
a second reaction product of a tetrazine amine and a second multi-arm polyethylene glycol comprising an amido succinic acid moiety.

2. The composition of claim 1, wherein the tetrazine amine comprises a methyltetrazine amine.

3. The composition of claim 1, further comprising a tissue adhesive moiety selected from the group consisting of catechol, hydrazide, and derivatives thereof, or a combination of two or more thereof.

4. The composition of claim 1, further comprising a cell.

5. The composition of claim 1, further comprising an active pharmaceutical ingredient.

6. The composition of claim 1, wherein the composition produces a hydrogel having a shear modulus from 1 kPa to 100 kPa.

7. The composition of claim 1, wherein the composition produces a hydrogel having a G′ from 1 kPa to 100 kPa.

8. The composition of claim 1, wherein the composition produces a hydrogel having a hydrolytic degradation half life from 5 days to 75 days.

9. (canceled)

10. (canceled)

11. The composition of claim 1, wherein the first multi-arm polyethylene glycol comprises eight arms or wherein the second multi-arm polyethylene glycol comprises four arms.

12. (canceled)

13. The composition of claim 11, wherein the four arms are conjugated to an unsubstituted tetrazine amine or wherein three of the four arms are conjugated to an unsubstituted tetrazine amine and one of the four arms is conjugated to a methyltetrazine amine or wherein two of the four arms are conjugated to an unsubstituted tetrazine amine and two of the four arms are conjugated to a methyltetrazine amine or wherein one of the four arms is conjugated to an unsubstituted tetrazine amine and three of the four arms are conjugated to a methyltetrazine amine or wherein the four arms are conjugated to a methyltetrazine amine.

14.-17. (canceled)

18. A wound dressing comprising:
a hydrogel formed from a hydrogel precursor, the hydrogel precursor comprising:
a first reaction product of a hydroxyl-terminated first multi-arm polyethylene glycol and a compound selected from the group consisting of a carbic anhydride, a norbornene acid, and a combination thereof, and
a second reaction product of a tetrazine amine and a second multi-arm polyethylene glycol comprising an amido succinic acid moiety.

19. The wound dressing of claim 18, wherein the tetrazine amine comprises a methyltetrazine amine.

20. The wound dressing of claim 18, further comprising a tissue adhesive moiety selected from the group consisting of catechol, hydrazide, and derivatives thereof, or a combination of two or more thereof.

21. The wound dressing of claim 18, wherein the hydrogel has a shear modulus from 1 kPa to 100 kPa.

22. The wound dressing of claim 18, wherein the hydrogel has a G′ from 1 kPa to 100 kPa.

23. The wound dressing of claim 18, wherein the hydrogel has a hydrolytic degradation half life from 5 days to 75 days.

24. (canceled)

25. (canceled)

26. The wound dressing of claim 18, wherein the first multi-arm polyethylene glycol comprises eight arms or wherein the second multi-arm polyethylene glycol comprises four arms.

27. (canceled)

28. The wound dressing of claim 26, wherein the four arms are conjugated to an unsubstituted tetrazine amine or wherein three of the four arms are conjugated to an unsubstituted tetrazine amine and one of the four arms is conjugated to a methyltetrazine amine or wherein two of the four arms are conjugated to an unsubstituted tetrazine amine and two of the four arms are conjugated to a methyltetrazine amine or one of the four arms is conjugated to an unsubstituted tetrazine amine and three of the four arms are conjugated to a methyltetrazine amine or wherein the four arms are conjugated to a methyltetrazine amine.

29.-32. (canceled)

33. A method for manufacturing a wound dressing comprising:
forming a composition comprising a hydrogel precursor, the hydrogel precursor comprising
a first reaction product of a hydroxyl-terminated first multi-arm polyethylene glycol and a compound selected from the group consisting of a carbic anhydride, a norbornene acid, and a combination thereof; and
a second reaction product of a tetrazine amine and a second multi-arm polyethylene glycol comprising an amido succinic acid moiety;
allowing the tetrazine amine of the second reaction product to react with the first reaction product to form the hydrogel, thereby forming a hydrogel; and
applying the hydrogel to a support, thereby forming the wound dressing.

34. The method of claim 33, further comprising
applying the composition comprising the hydrogel precursor to a wound prior to allowing the tetrazine amine of the second reaction product to react with the first reaction product to form the hydrogel; and
covering the composition with the support.

35. (canceled)

36. A method for treating a wound in a subject in need thereof, the method comprising:
forming the composition of claim 1;
applying the composition to the wound; and
allowing the tetrazine amine of the second reaction product to react with the first reaction product to form the hydrogel, thereby treating the wound.

37.-39. (canceled)

40. A method for encapsulating a drug, a cell, or both a drug and a cell, the method comprising:
forming the composition of claim 1;
mixing the drug, the cell, or both the drug and the cell, with the composition; and
allowing the tetrazine amine of the second reaction product to react with the first reaction product to form the hydrogel, thereby encapsulating the drug, the cell, or both the drug and the cell.
